(19) United States
(12) Patent Application Publication
(10) Pub. No.: US 2006/0094108 A1
(43) Pub. Date: May 4, 2006

(54) Thermal cycler for microfluidic array assays

(76) Inventors: Karl Yoder, Stoneham, MA (US); Colin Brenan, Marblehead, MA (US); John Linton, Lincoln, MA (US); Leila Hasan, Brookline, MA (US); Robert Ellis, Burlington, MA (US); Arrin Katz, Cambridge, MA (US); Tom Morrison, Winchester, MA (US); Jorge Fonseca, Boston, MA (US)

Correspondence Address: BROMBERG & SUNSTEIN LLP, 125 SUMMER STREET, BOSTON, MA, 02110-1618, US

(21) Appl. No.: 11/227,425

(22) Filed: Sep. 15, 2005

Related U.S. Application Data
(63) Continuation-in-part of application No. 10/744,580, filed on Dec. 22, 2003.
(60) Provisional application No. 60/610,033, filed on Sep. 15, 2004.
(60) Provisional application No. 60/434,988, filed on Dec. 20, 2002.
(60) Provisional application No. 60/461,559, filed on Apr. 9, 2003.
(60) Provisional application No. 60/528,461, filed on Dec. 10, 2003.
(60) Provisional application No. 60/461,556, filed on Apr. 9, 2003.

(51) Int. Cl. C12M 1/34

(52) U.S. Cl. 435/287.2; 435/293.1; 435/303.1

(57) ABSTRACT

A system for thermal cycling a plurality of samples. The system includes a case having a fluid-tight cavity defining an interior volume. A microfluidic array is disposed in the interior volume, the array including a sheet of material having a pair of opposed surfaces, a thickness, and a plurality of through-holes running through the thickness between the surfaces. A thermal cycler having at least one thermally controlled surface is adapted to thermally contact the case.

### CROSS REFERENCE TO RELATED APPLICATIONS

This application claims priority from U.S. Provisional Application Ser. No. 60/610,033, filed Sep. 15, 2004, entitled “Thermal Cycler for Microfluidic Array Assays.” This application is also a continuation-in-part of U.S. patent application Ser. No. 10/744,580, filed on Dec. 22, 2003, entitled “Assay Apparatus and Method Using Microfluidic Arrays,” which in turn claims priority from U.S. provisional patent application Ser. No. 60/434,988, entitled “Chip Temperature Cycling,” filed Dec. 20, 2002; U.S. provisional patent application Ser. No. 60/461,559, entitled “Immobilized Probe Nanotiter Array,” filed Apr. 9, 2003; U.S. provisional patent application No. 60/528,461, entitled “Improved Selective Ligation and Amplification Assay” filed Dec. 10, 2003; and U.S. provisional patent application Ser. No. 60/461,556, entitled “High-Density Microfluidic Thermal Cycling with Stackability,” filed Apr. 9, 2003. Each of these patent applications described in this paragraph is hereby incorporated by reference, in its entirety.

### TECHNICAL FIELD

The present invention relates to devices and methods for assaying samples in nanoliter volumes, potentially for achieving high throughput screening and for other purposes where the ability to assay low-volume samples at high densities is desired.

### BACKGROUND ART

The survival, growth and differentiation of a cell in normal and diseased states is reflected in altered patterns of gene expression and the ability to quantitate transcript levels of specific genes is central to any research into gene function. The recent completion of the human genome sequence and the emergence of molecular medicine has increased the need for higher throughput techniques to quantitate levels of RNA across many hundreds of genes and thousands of samples. Faced with this challenge, oligonucleotide and cDNA microarrays have emerged as the leading quantitative tool for analyzing transcription levels in many thousands of genes simultaneously. Despite this apparent success, it is well-established microarray data is fraught with errors from a variety of sources with the greatest contribution from the platform itself.

The real-time polymerase chain reaction (rt-PCR) is the standard by which the quality of microarray data is judged and validated. PCR itself is a high fidelity process for replicating a specific DNA sequence at levels down to a single molecule. This analytical versatility has made PCR an indispensable component of many bioanalytical methods and ubiquitous in modern biology. PCR is a temperature-modulated, enzymatic amplification for in vitro exponential replication of a nucleic acid sequence (target) defined by a pair of oligonucleotide sequences (primers) hybridized to their sequence complement. Kinetic or real-time PCR quantifies the number of template DNA copies by calibration of the fluorescent amplification signal with copy number. When the amplification signal reaches a level significantly above background, the fluorescence or cycle threshold (CT) is recorded and converted into template copy number based on a calibrated standard curve for that gene. RNA quantification requires reverse transcription of RNA into cDNA prior to application of the real-time PCR method.

PCR is a solution-phase assay carried out in 96- or 384-well microplates and scaling PCR to achieve higher throughputs with conventional technology is neither cost effective nor efficient. Consequently, it is therefore natural to consider if a larger number of PCR assays could be implemented simultaneously in smaller reaction volumes without compromising data quality or in other words, to combine the parallelism of a microarray with the quantification, sensitivity, dynamic range and specificity of qPCR in a single microfluidic device for high throughput transcription analysis.

Miniaturization of PCR reaction volumes to less than a microliter lowers consumption of expensive reagents and decreases amplification times from the reduced thermal mass of the reaction volume. It confers flexibility in selection of a strategy to scale analytical throughput, either by a fast serial or parallel array processing approach. These attributes must be balanced against the requirement the quality of data from a low volume PCR system equal that from larger volume reactions, typically 5-10 [symbol] L, in a microplate. A critical challenge in reaching this level of performance is the physical isolation of the reaction volumes to prevent evaporation and fluidic cross-talk between adjacent containers during thermal cycling and loading of sample and primers. Equally important are facile methods for liquid transfer of primer pairs, samples and PCR reagents between individual microcontainers and wells in a microplate without cross-contamination. Another factor impacting PCR assay quality in reduced volumes is the increased surface area-to-volume ratio. Surface interactions biasing PCR chemistry and kinetics can be mitigated by engineered coatings of the wetted surface for minimized reactivity or reformulation of the PCR by inclusion of compensating surface blocking agents.

Smaller volumes benefit from faster thermal cycling than larger volumes because the high surface area-to-volume ratio facilitates rapid heat transfer. Fabrication of microwell structures in high thermal conductivity, low specific heat materials like silicon or metal enable shorter thermal cycle times than those in standard microplate thermoplastics having a low thermal conductivity and a high specific heat.

Strategies for increasing PCR throughput and minimizing cost typically follow a two-fold approach: decrease the reaction volume required for amplification and increase the number of reactions performed over a given time. Parallel microfluidic assay arrays is one way to implement this strategy and one example of such an array is the Living Chip™ marketed by Biotrove, Inc. of Woburn, Mass. In function and purpose, the Living Chip™ is similar to 96- and 384-well microtiter plates currently used in high-throughput screening and diagnostics. However, the approximately 33 nl sample volume held by each sample well in the Living Chip™ is roughly 2000 times less than that in a 96-well plate, and 200 times less than a 384-well plate.

FIG. 1 shows a cut away view of a typical microfluidic array of through-holes. Such an array is described, for example, in U.S. Pat. No. 6,387,331 and U.S. Patent Application 20020094533, the contents of which are incorporated herein by reference. The sample array 10 includes a sheet of material 14 having a pair of opposed surfaces and a thickness. The sheet of material 14 may be a platen, otherwise referred to herein as a chip, and may be made of, for example, conductive silicon, or other types of rigid materials, such as metal, glass, or plastic. A large number of through-holes 12 (up to 3,072 through-holes at a density of 2 through-holes/mm in the present embodiment) run through the thickness from one of the surfaces to the other opposing surface (not shown).

The sample array 10 typically may be from 0.1 mm to more than 10 mm thick; for example, around 0.3 to 1.52 mm thick, and commonly 0.5 mm. Typical volumes of the through-holes 12 may be from 0.1 picoliter to 1 microliter, with common volumes in the range of 0.2-100 nanoliters, for example, about 33 nanoliters. Capillary action or surface tension of the liquid samples may be used to load the sample through-holes 12. For typical chip dimensions, capillary forces are strong enough to hold liquids in place. Chips loaded with sample solutions can be waved around in the air, and even centrifuged at moderate speeds without displacing samples.

To enhance the drawing power of the through-holes 12, the target area of the receptacle, interior walls 13, may have a hydrophilic surface that attracts a liquid sample. It is often desirable that the surfaces be bio-compatible and not irreversibly bind biomolecules such as proteins and nucleic acids, although binding may be useful for some processes such as purification and/or archiving of samples. Alternatively, the sample through-holes 12 may contain a porous hydrophilic material that attracts a liquid sample. To prevent cross-contamination (crosstalk), the exterior planar surfaces of chip 10 and a layer of material 15 around the openings of sample through-holes 12 may be of a hydrophobic material. Thus, each through-hole 12 has an interior hydrophilic region bounded at either end by a hydrophobic region.

The use of through-holes 12, as compared to closed-end well structures, reduces the problem of trapped air inherent in other microplate structures. The use of through-holes together with hydrophobic and hydrophilic patterning enables self-metered loading of the sample through-holes 12. The self-loading functionality helps in the manufacture of arrays with pre-loaded reagents, and also in that the arrays will fill themselves when contacted with an aqueous sample material.

When conducting PCR on the microfluidic array a series of heating and cooling cycles is used to replicate a small amount of DNA into a much larger amount. Thermal cyclers, such as a Peltier device, may be used to generate such a series of heating and cooling cycles. Implementing the method of real-time PCR requires the fluorescence from each reaction container (or containers) to be recorded at a pre-determined temperature in each heating and cooling cycle. To ensure proper thermal cycling of the microfluidic array in implementing the real-time PCR method, various issues arise. These include: preventing sample loss and/or contamination; proper placement and positioning of one or more microfluidic sampling arrays onto the thermal cycler to enable accurate and precise recording of the fluorescence emitted from each through-hole simultaneously of the microfluidic sampling array; recording the fluorescence from many through-holes simultaneously accurately and precisely; coordinating the thermal cycling with the recording of fluorescence in an automated system; optimizing thermal contact between the microfluidic sampling array and the thermal cycler; and preventing leakage of any evaporated fluids, so as to prevent, for example, condensation on any optical components within the system or inhibition of the PCR reaction in the microfluidic array.

### SUMMARY OF THE INVENTION

In a first embodiment of the invention there is provided a system for holding at least one of sample and reagent for analysis. The system includes a pair of parallel covers. At least one of the pair of parallel covers is light transmissive, of which pair a light transmissive cover forms a top, and of which pair the other forms a bottom. A frame is disposed between the covers to define, in relation to the covers, an interior volume. The frame and the covers are associated with one another to form a case that is substantially tight to liquids. A microfluidic array is disposed in the interior volume. The array includes a sheet of material having a pair of opposed surfaces, a thickness, and a plurality of through-holes running through the thickness between the surfaces. The through-holes contain at least one of sample and reagent.

In accordance with another embodiment of the invention, a system for holding at least one of sample and reagent for analysis is presented. The system includes a pair of parallel covers, at least one of which is light transmissive, and of which pair a light transmissive cover forms a top, and of which pair the other forms a bottom. A frame is disposed between the covers to define, in relation to the covers, an interior volume. The frame and the covers are associated with one another to form a case. The case includes a sealable opening, which when sealed renders the case substantially tight to liquids. A microfluidic array is disposed in the interior volume and is removable via the opening. The array includes a sheet of material having a pair of opposed surfaces, a thickness, and a plurality of through-holes running through the thickness between the surfaces. The through-holes containing at least one of sample and reagent.

In accordance with still another embodiment of the invention, a method of conducting an assay on a plurality of samples is presented. A microfluidic array is provided. The array includes a sheet of material having a pair of opposed surfaces, a thickness, and a plurality of through-holes running through the thickness between the surfaces. Each of the through-holes contains one of the samples and at least one reagent providing an optical effect for assay purposes. The array is place in a case that is substantially tight to liquids. The case includes a pair of parallel covers, at least one of which is light transmissive, and of which pair a light transmissive cover forms a top, and of which pair the other forms a bottom. A frame is disposed between the covers to define, in relation to the covers, an interior volume for receiving the array. The corresponding sample in each of the through-holes is permitted to react with the at least one reagent therein. A measurement is obtained, through the top cover, for each through-hole, of the optical effect associated therewith and the measurement is used to provide assay results for the corresponding sample therein.

In various embodiments related to the invention as described herein, a spacer means is provided for ensuring space between at least one of the covers of the case and at least a portion of the array. The top cover and the spacer means may be dimensioned to provide a distance of less than 0.5 mm from an upper surface of the top cover to a proximate surface of the array. The spacer means may include a plurality of beads or posts affixed to one of (i) the array and (ii) at least one of the covers, and/or an increase in thickness of the array over a defined set of locations thereof. One or more positioning guide rails may be affixed to at least one of (i) the frame and ii) at least one of the covers. The array may include a recess at an opening of each through-holes, the recess preventing fluid in each through-hole from coming into contact with a cover to which each such through-hole is proximate. The dimensions of the case may be approximately 25×76×<2 mm, such that the case has the approximate size and shape of a microscope slide. The frame of the case may includes walls defining a hole, the hole filled with a self-sealing material, such as grease, and the frame may be a gasket that can be penetrated by a syringe. The frame and the covers may be coupled together to form the case by an epoxy or other adhesive. In various embodiments, the frame may be, or include, an adhesive gasket, and/or a compression gasket.

In further related embodiments to the invention described herein, a funnel guide may be coupled to the case, the array capable of being inserted into the case by passing the array through the funnel guide and an opening of the case. The funnel guide may be removably attached to the case. The funnel guide may include walls defining a slit, the array capable of being passed through the slit. Liquid may be substantially prevented from passing through the slit in the absence of the array due to, for example, surface energy. The walls defining the slit may be capable of being deformed to allow the array to pass through the slit, and may be made, for example, of plastic. The slit may be capable of being opened and closed. The funnel guide may include brushes for spreading of the at least one of sample and reagent. The at least one cover of which is light transmissive may be coated with a hydrophilic layer to prevent fogging. At least one of the frame and the covers may includes a hydrophilic strip for promoting spreading of sample during array loading. At least one of the array and the case may include an identifier, such as a barcode.

Another embodiment of the present invention includes a thermal cycling device and corresponding method. A fluid delivery system develops a flow of controlled-temperature fluid, which may be selectable between a first controlled temperature and at least a second controlled temperature. A sample plate cartridge has a cavity for holding a high-density microfluidic sample plate. A cycling head holds the sample plate cartridge and delivers the flow of fluid over the sample plate cartridge.

A further embodiment may include a thermal sensor for sensing temperature of the flow of fluid. The sample plate cartridge may also include at least one transparent cover over the sample plate, and the cycling head may include at least one transparent window arranged for imaging of samples in the sample plate. A Peltier device may be associated with the cycling head for controlling temperature of the fluid.

The cycling head may be adapted for vertical orientation of the sample plate cartridge. The sample plate cartridge may include a guide rail arrangement for holding the sample plate, and/or may be capable of holding a plurality of sample plates. Alternatively or in addition, the cycling head may include a guide rail arrangement for holding the sample plate cartridge.

The sample plate cartridge or the cycling head may be adapted to deliver a laminar flow of fluid over the sample plate cartridge. The cycling head may include a flow regulator for promoting uniform flow of fluid over the sample plate cartridge. The flow regulator may include a flow restrictor or flow inlet cavity in the cycling head upstream of the sample plate cartridge. A volume of fluid that is immiscible with the sample such as (for aqueous samples) a perfluorinated hydrocarbon liquid may be provided in the sample plate cartridge cavity for covering an inserted sample plate.

In an embodiment, the sample plate may have a top surface and a bottom surface which are connected by a plurality of through-holes, and the sample plate cartridge may have an associated top cover and bottom cover. In such an embodiment, the sample plate cartridge and the cycling head may be adapted so that the flow of fluid is delivered over both the top cover and the bottom cover.

Another embodiment of the present invention is directed to a microfluidic array which includes a platen having a high-density microfluidic array of through-holes. A biocompatible and/or hydrophilic coating is coupled to walls of at least one through-hole well of the array. Encapsulated in the coating is a primer for amplifying a nucleotide sequence of a sample introduced into the through-hole. The coating may be covalently bonded or dried to the interior walls of the through-holes. The biocompatible material may be a polymer such as polyethylene glycol. The primer may be for PCR assaying. A second layer of polymer may be added to the top of the coating. In various embodiments, the array may include a layer of hydrophobic material around the opening of each through-hole, so as to isolate each through-hole from other through-holes. The platen may be arranged for stacking with another platen to promote a desired chemical reaction in each through-hole.

In various embodiments, a sample containing nucleic acid can be introduced to a sample platen that includes an array having capture probes, so as to form a hybridized array of samples. Then, PCR sequencing can be performed on the hybridized array. In some embodiments, this may involve providing a second reagent platen having a high-density microfluidic array of through-holes, in which each through-hole contains a volume of PCR reagent, and in which the reagent platen has a structural geometry that corresponds to the sample platen. Then, one platen can be stacked on top of the other so as to deliver PCR reagent to samples in the hybridized array. In various embodiments, the hybridized array may be washed, prior to stacking, with a buffer to remove on-specifically bound nucleic acids.

Another representative embodiment of the present invention includes a microfluidic array for thermal cycling. A platen has a layer of hydrophobic material surrounding the openings of through-holes of the array that include a biocompatible and/or hydrophilic coating, wherein at least one through-hole includes a covalently or non-covalently immobilized nucleic acid component for assaying. The nucleic acid component may be immobilized in a hydrophilic polymer and/or a melting polymer that melts during assaying so as to release the nucleic acid component into solution in the at least one through-hole. For example, the polymer may be based on polyethylene glycol (PEG). The nucleic acid component may be a primer or a probe for polymerase chain reaction (PCR) assaying.

A corresponding method of biochemical assaying starts by loading a polymer solution containing a nucleic acid into at least one through-hole in an high-density microfluidic array of through-holes, the array having a layer of hydrophobic material surroundings the openings of the through-holes, and each through-hole containing a hydrophilic material. The solution is then dried so that a nucleic acid component is immobilized within the at least one through-hole.

The method may further include loading a nucleic acid target component into the at least one through-hole, and then thermal cycling the array and performing a PCR assay. The loading may be based on dipping the array into a solution containing the nucleic acid target component, and then withdrawing the array from the solution. Alternatively, the nucleic acid target component may be pippetted into the at least one through-hole, or a drop of solution containing the nucleic acid target component may be moved relative to the opening of the at least one through-hole. The thermal cycling may include developing a flow of controlled-temperature fluid; loading the array into a sample plate cartridge having a cavity for holding a high-density microfluidic sample plate; and delivering the flow of controlled-temperature fluid over the sample plate cartridge.

In accordance with another embodiment of the invention, a biochemical assay structure and method includes a chip having a microfluidic array of through-holes. The through-holes are adapted for: capture of one or more targets of interest from a liquid sample introduced into the individual through-hole; and chemical processing of the captured one or more targets.

In related embodiments of the invention, the target capture may be based on a capture structure immobilized within the individual throughholes, such as a nucleic acid probe. The capture structure may be a protein, an antibody, and/or an aptamer. The capture structure may be covalently immobilized. The capture structure may be selected from antibodies, proteins, peptides, peptide nucleic acids, and oligonucleotides. The chemical processing may include amplification of the captured one or more targets. The amplification may include at least one of polymerase chain reaction (PCR) amplification and reverse transcription. The chemical processing may include detection of a signal from the captured one or more targets. The chemical processing may be specific to the captured one or more targets. The structure may be adapted to perform lysis of a target pathogen, or to perform ELISA analysis. The individual through-holes may include a layer of wax containing at least one reagent for the target capture or chemical processing. The wax may include polyethylene glycol (PEG), and/or have a melting point above 40° C. The individual through-holes may include a plurality of layers of wax, at least one of the layers containing the at least one reagent. Each layer of wax may have a different melting point and/or a different reagent. The surfaces of the through-holes may be bio-compatible to avoid binding bio-molecules.

In further related embodiments of the invention, the assay structure and/or method may further include a first chip layer having a microfluidic array of through-holes and a second chip layer having a microfluidic array of through-holes. The first chip layer and the second chip layer are fixedly coupled such that the through-holes of each are aligned. The individually aligned through-holes may be, for example, adapted for the target capture and the chemical processing. The first and second chip layers may be coupled by an adhesive, screws, bolts, rivets, and/or clamps.

In accordance with another embodiment of the invention, a method of conducting an assay on a plurality of samples includes performing an assay at each sample site in a sample array having greater than 100 sample sites. Each assay provides an optical effect. Each of the sample sites simultaneously imaged to produce imaging data pertinent to the optical effect of each site.

In related embodiments of the invention, the sample array has greater than 500 sample sites, or greater than 1600 sample sites. Performing the assay may include performing replication cycles by Polymerase Chain Reaction (PCR). Imaging may include simultaneously imaging each sample site during each replication cycle. Each sample site may be simultaneously illuminated using one or more LEDs. The method may further include analyzing the imaging data.

In accordance with another embodiment of the invention, a method of conducting an assay on a plurality of samples includes performing an assay at each of a plurality of sample sites in a sample array, the sample array having a sample site density greater than one sample site per 20 mm². Each assay provides an optical effect. Each of the sample sites is simultaneously imaged to produce imaging data pertinent to the optical effect of each site.

In related embodiments of the invention, performing the assay includes performing replication cycles by Polymerase Chain Reaction (PCR). Imaging may include simultaneously imaging each sample site during each replication cycle. Each sample site may be simultaneously illuminated using one or more LEDs. The method may further include analyzing the imaging data.

In accordance with another embodiment of the invention, a method of conducting an assay on a plurality of samples includes performing an assay at each of a plurality of sample sites in a sample array. Each assay provides an optical effect. Each sample site is simultaneously illuminated using one or more colored LEDs. Furthermore, each of the sample sites is simultaneously imaged to produce imaging data pertinent to the optical effect of each site.

In related embodiments of the invention, performing the assay may include performing replication cycles by Polymerase Chain Reaction (PCR). Each sample site may be simultaneously imaged during each replication cycle. The method may further include analyzing the imaging data.

In accordance with another embodiment of the invention, a system for conducting an assay on a plurality of samples includes a case having a fluid-tight cavity defining an interior volume. A microfluidic array is disposed in the interior volume, the array including a sheet of material having a pair of opposed surfaces, a thickness, and a plurality of through-holes running through the thickness between the surfaces. A thermal cycler is adapted to thermally contact the case.

In related embodiments of the invention, the thermal cycler may be a flat block having at least one thermally controlled surface for thermally contacting the case. The thermally controlled surface may be flat and may have regions capable of being illuminated and imaged. The illuminated and imaged regions may be at least the same extent as the microfluidic array. The thermal block may include markings to delineate positioning of the microfluidic array relative to the illuminated and imaged area, and/or a positioning mechanism for positioning the microfluidic array at a fixed position on the thermally controlled surface. The positioning mechanism may include an indention on the thermally controlled surface. The indentation may include a graded surface, such that the microfluidic array can be slid into the indentation. The positioning mechanism may include a raised region against which the microfluidic array is placed to position it within the illuminated and imaged region. A heat transfer pad may be positioned between the case and the thermally controlled surface.

In further related embodiments, the system may include an illumination source, the illumination source for illuminating the microfluidic array at least one specific wavelength. The illumination source may be capable of illuminating each of the through-holes simultaneously. The illumination source may include at least one LED. The illumination source may include a plurality of LEDs oriented relative to the microfluidic array and camera such that substantially no specular reflections from the microfluidic array enter the camera. The at least one LED may be filtered by an excitation filter.

In still further related embodiments, an imaging device may simultaneously or sequentially image each of the through-holes to provide imaging data. The imaging device may be, for example, a camera or a scanner. The illumination source may include at least two illuminations sources symmetrically positioned off-axis from the imaging device with reference to the array. The system may further include a processor for processing the imaging data.

In yet further embodiments of the invention, the case may include a pair of parallel covers, at least one of which is light transmissive, of which pair a light transmissive cover forms a top, and of which pair the other forms a bottom. A frame disposed between the covers defines, in relation to the covers, an interior volume, the frame and the covers associated with one another to form the case. An immersion fluid may be disposed in the interior volume.

In further related embodiments, the thermal cycler may include a deck, which may be a smooth surface, for placing the microfluidic array prior to loading or removal from the thermal block. The deck may include an edge onto which the microfluidic array can be placed, whereupon the microfluidic array can be rotated onto the thermally controlled surface of the flat block. The thermal cycler may include a finger element for pressing the microfluidic array against the thermal block. The finger element aids in improving thermal contact between the case and flat block and preventing the case from moving relative to the illuminated and imaged area during temperature cycling. The finger element may be flexible. The finger element may be coated with an insulating material. The thermal cycler may include a lid assembly. The lid assembly may include the finger element. The fingers may touch the microfluidic array before the lid assembly is closed, such that a force is applied to the microfluidic array when the lid assembly is closed. The finger element may not be part of the lid assembly and may be placed on the case prior to closing the lid. The finger element may contact the case at one or more points.

In still further related embodiments, the lid assembly may include a gasket for sealing the lid assembly when closed. The lid assembly may include one or more stops that limit the opening or closing of the lid assembly. The lid assembly may include an optical window that may have a lens. One or more temperature control elements may measure the temperature of the thermal block, and control the temperature of the thermal block as a function of the temperature. The temperature control unit may provide Proportional, Integral and Derivative (PID) temperature control. The temperature control unit may include an offset for compensating between differences in heating rates of the thermal block and the microfluidic array. The temperature control unit may include a slow ramp mode. The array may have greater than 100 through-holes and/or a through-hole density greater than one through-hole per 20 mm².

In yet further embodiments of the invention, the system may include an enclosure into which the thermal cycler, an imaging device and an illumination device are positioned. The enclosure may be capable of being substantially light-tight when performing imaging. The enclosure may include a door for loading and removal of the microfluidic array. The system may include an illumination control element for preventing the illumination source from operating when the door is open.

In accordance with another embodiment of the invention, a method of thermal cycling a plurality of samples includes holding a microfluidic array in a fluid-tight cavity in a case, the array including a sheet of material having a pair of opposed surfaces, a thickness, and a plurality of through-holes running through the thickness between the surfaces. The case is placed in thermal contact with a thermally controlled surface.

In accordance with another embodiment of the invention, a system includes a case having a fluid-tight cavity defining an interior volume. A microfluidic array is disposed in the interior volume, the array including a sheet of material having a pair of opposed surfaces, a thickness, and a plurality of through-holes running through the thickness between the surfaces. The system further includes an illumination source for simultaneously illuminating each of the through-holes, and a camera for simultaneously imaging each of the through-holes to produce imaging data.

In related embodiments of the invention, the illumination source includes at least one Light Emitting Diode (LED). The at least one LED may be a colored LED. An excitation filter may filter the at least one LED. At least one LED may be symmetrically positioned off-axis from the camera with reference to the array. The camera may be one of a Charge-Coupled Device (CCD) or Complimentary Metal-oxide Semiconductor (CMOS) camera. The system may include an emission filter for filtering light entering the camera. The array may have greater than 100 through-holes, greater than 500 through-holes, or greater than 1600 through-holes. The array may have a through-hole density greater than one through-hole per 20 mm², or greater than one sample sites per 0.25 mm². In various embodiments, the system may further include a processor for analyzing the imaging data.

In accordance with another embodiment of the invention, a system for holding at least one of sample and reagent for analysis includes a pair of parallel covers, at least one of which is light transmissive, of which pair a light transmissive cover forms a top, and of which pair the other forms a bottom. A frame is disposed between the covers to define, in relation to the covers, an interior volume, the frame and the covers associated with one another to form a case. The case has a sealable opening, such opening when sealed rendering the case substantially tight to liquids. A microfluidic array is disposed in the interior volume and removable via the opening. The array includes a sheet of material having a plurality of sample sites, the sample sites containing at least one of sample and reagent.

In related embodiments of the invention, the array may include a hydrophobic surface surrounding the openings of each sample site. The sample sites may include a hydrophilic surface that attracts the at least one of sample and reagent. The sheet may have a pair of opposed surfaces and a thickness, and the sample sites include a plurality of through-holes running through the thickness between the surfaces. The sample sites may include a plurality of closed-ended wells. At least one cover of which is light transmissive may be coated with a hydrophobic layer to prevent fogging. The array may include a recessed opening at each sample site, the recess preventing fluid in each sample site from coming into contact with a cover to which each such sample site is proximate. The system may further include one of a UV curable sealent and a grease for sealing the opening. The frame and the covers may be coupled together to form the case by at least one of an epoxy or other adhesive. The frame may be, or include, an adhesive gasket or a compression gasket. The frame may be puncturable and include includes walls defining a hole, the hole filled with a self-sealing material, which may be, for example, a grease. The system may further include a funnel guide coupled to the case, the array capable of being inserted into the case by passing the array through the funnel guide and the opening. The funnel guide may be removably attached to the case. The funnel guide may includes walls defining a slit, the array capable of being passed through the slit. Liquid may be substantially prevented from passing through the slit in the absence of the array due to, at least in part, surface energy. The walls defining the slit may be capable of being deformed to allow the array to pass through the slit. The funnel guide may include brushes for spreading of the at least one of sample and reagent. At least one of the frame and the covers may include a hydrophilic strip for promoting spreading of sample during array loading.

### BRIEF DESCRIPTION OF THE DRAWINGS

The foregoing features of the invention will be more readily understood by reference to the following detailed description, taken with reference to the accompanying drawings, in which:

FIG. 1 is a diagram illustrating a typical sample array of through-holes according to prior art;

FIG. 2 is an exploded perspective view of a case for a sample array, in accordance with an embodiment of the present invention;

FIG. 3(a) is a diagram illustrating a top view of a case that includes a U-shaped frame with centering guide rails, in accordance with an embodiment of the invention;

FIG. 3(b) is a diagram illustrating a side view of the case shown in FIG. 3(a), in accordance with an embodiment of the invention;

FIG. 4 is a block diagram of a method for providing a system including an array, a case, and related components so as to permit a user to perform assays, in accordance with an embodiment of the invention;

FIGS. 5 through 16 are diagrams illustrating an embodiment by which a user may perform assays using the system described in connection with FIG. 2;

FIG. 5 and FIG. 6 are diagrams illustrating the addition of immersion fluid to a case, in accordance with an embodiment of the present invention;

FIG. 7 and FIG. 8 are diagrams illustrating the addition of sample to the case of FIG. 6, in accordance with an embodiment of the present invention;

FIGS. 9 and 10 are diagrams illustrating the insertion of a microfluidic array into the case of FIG. 8, in accordance with an embodiment of the present invention;

FIG. 11 is a diagram illustrating the removal of excess sample from the case of FIGS. 10, in accordance with an embodiment of the present invention;

FIGS. 12 and 13 are diagrams illustrating the application of a sealant to the case of FIG. 11, in accordance with an embodiment of the present invention;

FIG. 14 is a diagram illustrating the use of ultraviolet light to cure the sealant applied in the manner illustrated in FIG. 13, in accordance with an embodiment of the present invention;

FIG. 15(a) is a diagram illustrating a sealed case resulting from practice of the method of FIG. 14, in accordance with an embodiment of the present invention;

FIG. 15(b) is a diagram illustrating a top view of a sealed case that includes a grease lock, in accordance with an embodiment of the present invention;

FIG. 16(a) is a diagram illustrating the introduction of a sample into through-holes of a microfluidic array in accordance with an embodiment of the present invention in which turbulence is introduced into the case;

FIG. 16(b) is a diagram illustrating the introduction of a sample into through-holes of a nano-liter array in accordance with an embodiment of the present invention, in which the microfluidic array is rotated;

FIG. 17 is a diagram illustrating an embodiment of the present invention facilitating the introduction of sample into through-holes of a microfluidic array via a funnel, in accordance with an embodiment of the present invention;

FIG. 18 is a diagram illustrating use of the sealed case of FIG. 15 in a thermal cycler, and in a scanner, so as to provide data that is subject to analysis in analysis software, in accordance with an embodiment of the present invention;

FIG. 19 is a diagram illustrating a thermal cycling system, in accordance with an embodiment of the present invention;

FIGS. 20(a-c) are diagrams illustrating structural details of various specific cycling head embodiments, in accordance with various embodiments of the present invention;

FIG. 21 is a diagram illustrating a side view of a thermal cycling flat block, in accordance with an embodiment of the present invention;

FIG. 22 is a diagram illustrating an imaging system, in accordance with an embodiment of the present invention;

FIG. 23 is a diagram illustrating a transmission imaging system using one or more Light Emitting Diodes (LEDs), in accordance with an embodiment of the present invention;

FIG. 24 is a cross-sectional side view of a thermal cycler system, in accordance with one embodiment of the invention;

FIG. 25 is a perspective view of a thermal cycler with a lid assembly in the open position, in accordance with one embodiment of the invention;

FIG. 26 is a perspective view of the thermal cycler of FIG. 4 with the lid assembly in the closed position;

FIG. 27 is a perspective view of a lid assembly, in accordance with one embodiment of the invention;

FIG. 28 is an illustration of a thermal cycle with a lid assembly having a spring mechanism for securing the sample array, in accordance with one embodiment of the invention;

FIG. 29(a-b) is a diagram illustrating a through-hole of a microfluidic array that includes layers of various material, in accordance with an embodiment of the invention; and

FIG. 30 is a diagram illustrating a layered microfluidic array structure, in accordance with an embodiment of the invention.

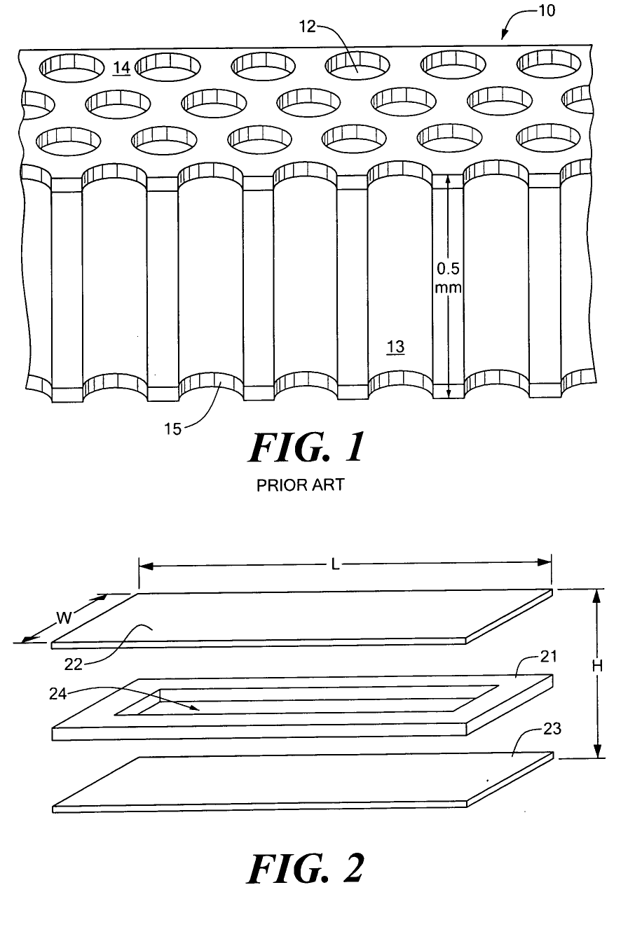

### DETAILED DESCRIPTION OF SPECIFIC EMBODIMENTS

Definitions. As used in this description and the accompanying claims, the following terms shall have the meanings indicated, unless the context otherwise requires:

“Target” may be any molecule, nucleic acid, protein, virus, cell, or cellular structure of interest.

“Microfluidic array” refers to any ordered structure for holding liquid samples of 1000 nanoliters or less.

Embodiments of the present invention are directed to devices and methods for assaying sample liquids using a microfluidic sample array. For example, various techniques for encasing, loading, stacking, thermal cycling and imaging of a microfluidic sample array are presented. Other embodiments of the present invention include adapting individual through-holes of the sample array for capture, chemical processing of captured targets, and/or multi-functional processing of liquid samples. Various examples and embodiments are discussed in detail below.

Encased Microfluidic Array

FIG. 2 is an exploded perspective view of a case for a microfluidic sample array, which may be include a plurality of through-holes and/or wells, in accordance with an embodiment of the present invention. The case includes a frame 21, a top 22, and a bottom 23 that, in operation, are placed in sealed relationship to one another such that the case is substantially tight to liquids, and in preferred embodiments, impermeable to low surface energy fluids that are immiscible with water, such as mineral oil or perfluorinated liquids. Under these conditions, the foregoing components define an interior volume 24, into which may be placed a microfluidic sample array.

At least one of the top 22 and the bottom 23 may be advantageously light transmissive, and in various embodiments both the top and the bottom are light transmissive. Light transmissivity of the top and/or the bottom facilitates optical reading of individual through-holes of the array when the array is placed in the interior volume 24 of the case. To prevent fogging, the at least one top 22 or bottom 23 may be coated with a hydrophilic layer.

In some embodiments it is desirable that the case of FIG. 2 have the approximate dimensions of a microscope slide, namely, 25 mm×75 mm×<2 mm (corresponding to dimensions W×L×H shown in FIG. 2) so that the case may be handled by microscope slide handling equipment. To facilitate automated handling of the case, it is desirably that the case be mechanically robust. Moreover, it is often useful to place an “encapsulation” in the interior volume with the microfluidic array. The term “immersion fluid” will be used interchangeably with the term “immersion fluid” to reflect that the encapsulation fluid may advantageously, but does not necessarily, assist in providing isolation between through-holes of the array, but may rather help to prevents evaporation of samples and maintain a uniform temperature throughout the array. This fluid is desirably immiscible with water and substantially unreactive with reactants and analytes that may be placed in through-holes of the array. Typical immersion fluids that may be used alone or in combination include, without limitation, mineral oil, silicon oil, and a perfluorinated hydrocarbon or mixture of perfluorinated hydrocarbons, such as perfluorinated alkane (such as Fluorinert from 3M, sold for use as electrical testing fluid), or perfluorinated polyether (available, for example, under the brands Fomblin® and Krytox®, from Solvay Solexis (Thorofare, N.J.) and DuPont (Wilmington, Del.) respectively, and sold for purposes including vacuum pump lubricants). In various embodiments, it is desirable that the immersion fluid have a specific gravity greater than 1. In various embodiments, the case is desirably sealed when subjected to assay conditions that may include thermal cycling and, potentially, chemical reactions, that may produce internal pressure changes, and the case is desirably dimensionally stable over the range of expected pressure change. It may be desirable that the immersion fluid remain a liquid over the temperature range of the assay which would require that it is substantially non-volatile at room temperature, have a freezing point that is less than room temperature and have a boiling point greater than the highest temperature used in an assay (typically 95° C. for PCR). The halogenated fluids typically permit less evaporation of the samples than the other fluids and are particularly useful for PCR.

As discussed in further detail below, in many instances it is desirable to form the case in such a way that one of its six sides remains open so as to permit insertion into the interior volume of the array and sealing after the array has been inserted. A convenient way of doing this is to make the frame 21 in a U-shape, for example, with the frame open along one side of its width to permit insertion of the array. After the array is inserted, the remaining leg of the frame (and open side of the case) may be sealed. Alternatively, a slot may be formed in one side of the frame that permits insertion of the array, which can then be sealed, or otherwise closed, after insertion of the array.

The frame 21, top 22, and/or bottom 23 may be coupled together to form the case by, without limitation, at least one of an epoxy or other adhesive. In various embodiments, the frame 21 may be implemented as a gasket (for example, of closed-cell acrylic foam) which may work under compression and/or be provided with adhesive on both sides to adhere to the top 22 and bottom 23, which may suitably be made on either top 22 or bottom 23 of glass, or a polycarbonate plastic. One of the top 22 or bottom 23 may be made of an opaque material such as a metal, with the other side permitting optical readout. The opaque part may be advantageously made from a heat conducting material such as stainless steel, which may be placed adjacent a heat source, such as a Peltier device, during thermal cycling.

The geometry of the case in relation to the array is often important to the design and implementation of the system. For example, the gap between the array and the case, and surface treatment on both sides of the array can affect: the ability to load the sample into the chip in situ; the formation and behavior of gas or vapor bubbles during thermal cycling; and whether the gas bubbles that may be generated can cause sample evaporation with resulting condensation of water vapor on the case or chip surfaces.

To ensure proper separation between the array and the case, the surfaces of the top 22 and the bottom 23 which face the interior volume 24 may be equipped with a spacing means such as shims, bumps, and or posts protruding from them so that the array does not contact the surfaces. Alternatively, the array itself may be provided with shims, bumps, and/or posts on its faces so that the sample does not contact the surfaces of the top 22 and bottom 23 that face into the interior volume 24. In various embodiments, spacing may be achieved by providing a mixture of glass beads in glue that is applied to select locations on the array. In other embodiments, the array may be fabricated with suitable spacing elements formed of the array material itself to provide any desired spacing between the bulk of the array and the inner facing portions of the top 22 and bottom 23.

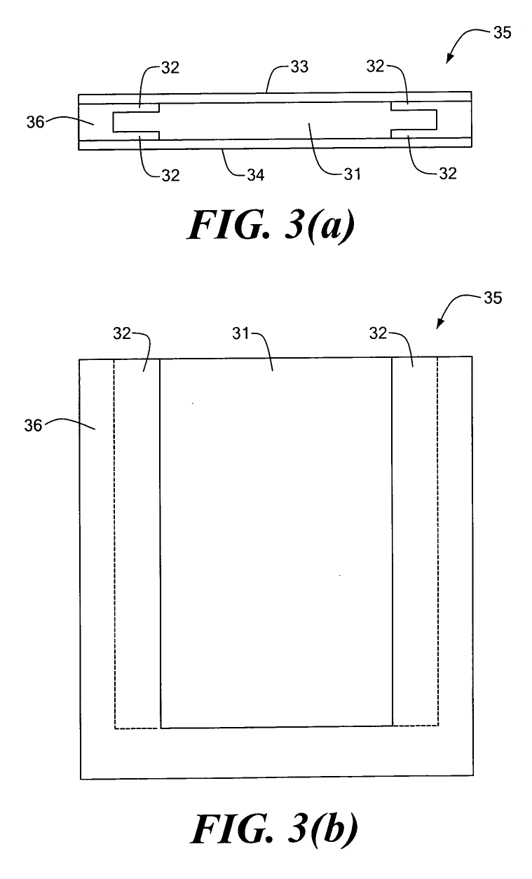

FIGS. 3(a) and 3(b) shows a top view and a side view, respectively, of a case 35 that includes a U-shaped frame 36 with centering guide rails 32, in accordance with one embodiment of the invention. In various embodiments, the centering guide rails 32 may be attached or integral to the covers 33, 34 or the frame 36, or both. The centering guide rails 32 securely hold the sides of an inserted array in between a left cover 33 and a right cover 34. In one specific embodiment, the through-holes of the array are held in position without touching either the left cover 33 or the right cover 34. The concept of left and right covers 33 and 34 suggests that the case 35 possesses a vertical orientation. In other embodiments, the case 35 may have a horizontal orientation (in which case the covers would correspond to the top 12 and bottom 13 of FIG. 2), or a hybrid orientation.

In illustrative embodiments of the invention, the case may include fill lines to indicate the level of encapsulating liquid. The fill lines may be silk screened or otherwise printed onto the case. Printed lines may also be used to mask fluorescent adhesive material along the rim of the case.

Preparing and Loading the Microfluidic Array

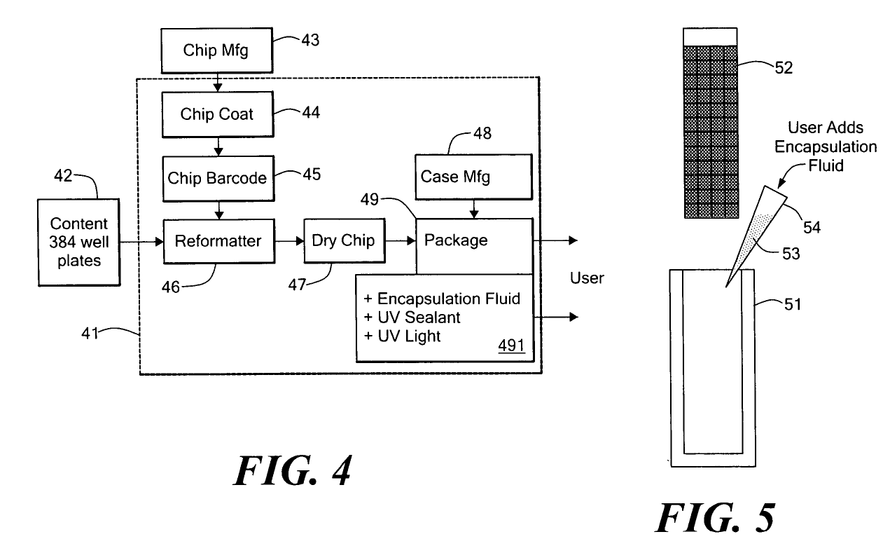

FIG. 4 is a block diagram of a method in accordance with the present invention for providing a system including a microfluidic array, a case, and related components so as to permit a user to perform assays using the system. The processes enclosed by dashed line 41 are typically performed by the supplier of the assay system. In process 42, the supplier is provided with content to be introduced into through-holes of the array, and here it is provided in a plate having 384 wells. The content may be reactants, and alternatively, or in addition, may include, for example, samples, standards, or analytes. Meanwhile, in process 43, the supplier is also provided with the array in a raw form as a sheet of material, for example, of silicon or steel in which through-holes have been formed. In process 44, the array is treated, for example with hydrophobic and hydrophilic material, and in process 45 appropriately barcoded. In process 46, the array is populated with the content derived from the plates obtained in process 42. In process 47, the array is dried in preparation for packaging which occurs in process 49. In process 48, meanwhile, a suitable case is prepared as discussed previously in connection with FIG. 2. In this circumstance, the case is prepared with an open side as discussed above. The user receives a system that includes the array, stored in the case, immersion fluid as discussed above, and an arrangement for sealing the case after the array has been further populated by the user. For example, the sealing arrangement may include a sealant that is activated by ultraviolet light, as well as a source for the ultraviolet light used to activate a sealant. The supplies of the fluid, sealant and light, are indicated by box 491.

FIGS. 5 through 16 are diagrams illustrating an embodiment by which a user may perform assays using the system described in connection with FIG. 4.

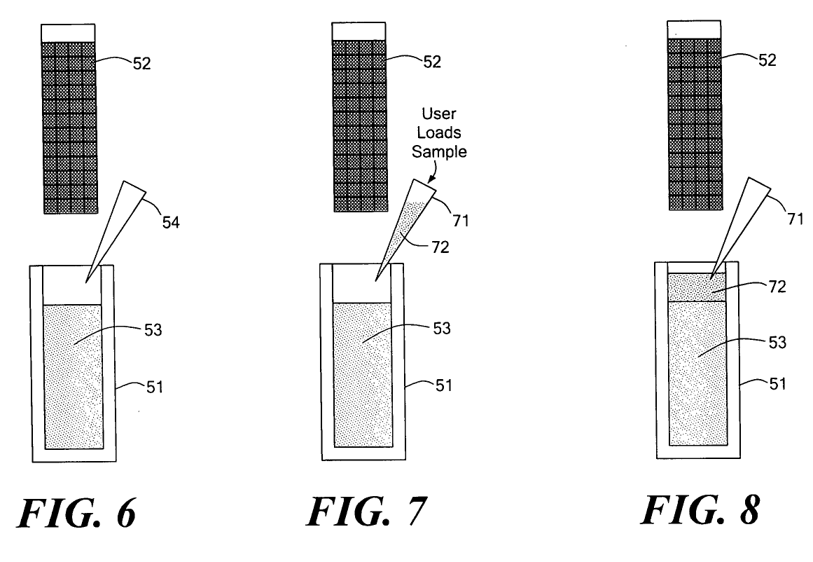

FIG. 5 and FIG. 6 are diagrams illustrating the addition of an immersion fluid 53 to a case 51, in accordance with an embodiment of the present invention. An array 52 is depicted outside of the case 51. In FIG. 5, immersion fluid 53 is provided in a dispenser 54, which may be, for example, a syringe or similar equipment. Using the dispenser 52, the immersion fluid is added to the case 51, as shown in FIG. 6.

FIG. 7 and FIG. 8 are diagrams illustrating the addition of sample 72 to the case 51 of FIGS. 5 and 6 after the immersion fluid 53 has already been added, in accordance with an embodiment of the present invention. In FIG. 7, the immersion fluid 53 is shown in the case 51, and a dispenser 71 (which may again be implemented as a syringe or similar device) is used to load sample 72 into the case 51. In FIG. 8, the sample 72, being aqueous based, is shown lying above the immersion fluid 53, which has a specific gravity greater than 1.

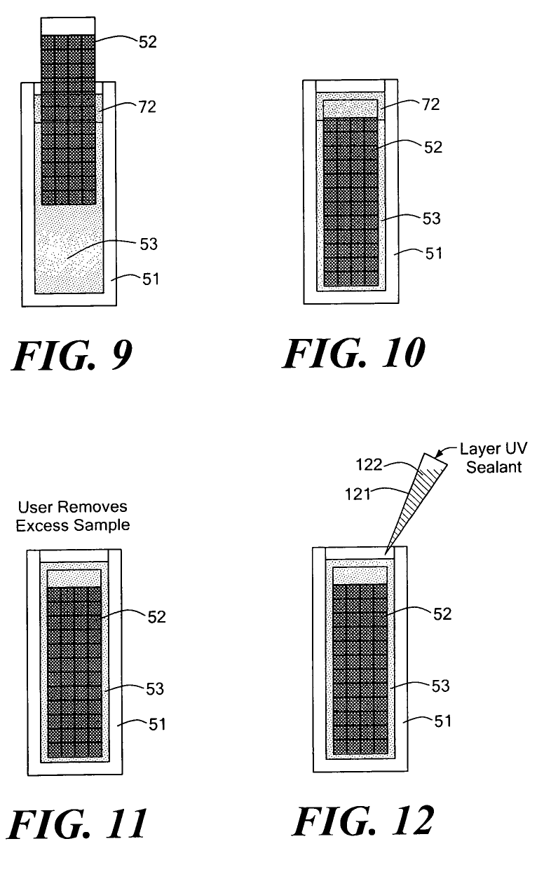

FIGS. 9 and 10 are diagrams illustrating the insertion of a microfluidic array 52 into the case 51 of FIGS. 5 and 6 in accordance with an embodiment of the present invention. In FIG. 9, the array has been inserted part way, and it can be seen that before any through-hole of the array 52 reaches the immersion fluid 53, it is passed through sample 72 where it may engage the sample 72. In FIG. 10, the array 52 has been fully inserted into the case 51, and all through-holes of the array have passed through the sample 72. At this point, the through-holes of the array 52 are fully populated.

After the array 52 has been full inserted into the case 51, any excess sample is removed. FIG. 11 is a diagram illustrating removal of excess sample (shown as item 72 in FIG. 10) from the case 51, in accordance with an embodiment of the present invention. Since the sample 72 lies on top of the immersion fluid 53, as shown in FIG. 10, the excess sample may be removed in a straightforward manner.

After the excess sample has been removed from the case 51 as shown in FIG. 11, the case 51 can be sealed. In various embodiments, the case 51 may undergo further processing prior to sealing. For example, the case may be thermally cycled before sealing, as described in more detail below. If kept in a vertical position throughout the analysis, sealing may be avoided entirely, although the case may be prone to spillage.

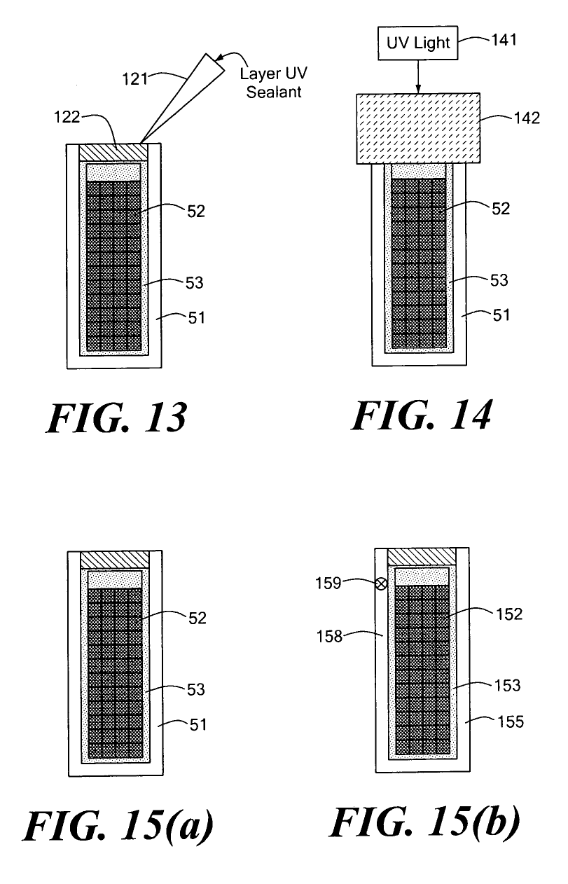

FIGS. 12 and 13 are diagrams illustrating the application of a sealant 122 to the case 51, in accordance with an embodiment of the present invention. A dispenser 121 may be used to dispense sealant 122 to the open side of case 51.

The sealant illustrated here is cured by exposure to ultraviolet light. Accordingly, FIG. 14 is a diagram illustrating the use of ultraviolet light to cure the sealant applied in the manner illustrated in FIGS. 12 and 13, in accordance with an embodiment of the present invention. Here an ultraviolet light source 141 provides ultraviolet light (illustrated schematically as item 142) to the sealant to cause it to be cured. Alternative sealants, which are not cured by ultraviolet light, may also be employed. In various embodiments, the sealant is a suitably thick and inert substance, such as a high vacuum grease. Suitable high vacuum greases may include silicone, and also perfluorinated polyether/PTFE substances, such as Fomblin® VAC™ 3, a perfluoropolymer based vacuum grease thickened with a PTFE thickener, from Solvay Solexis (Thorofare, N.J.). Alternatively, a suitable wax may be used in appropriate circumstances.

FIG. 15(a) is a diagram illustrating the case 51 after sealing. As an alternative to the loading arrangement just described, the array may be, placed in the case, and sample added to the case to fill the array, excess sample removed and then immersion fluid can be added through one or more open sides or injected directly through the frame material if it is a self-sealing material. To provide self-sealing properties, a gap in the frame material may be filled with a second material, such as vacuum grease. In such a case, immersion fluid may be dispensed through the grease using a syringe, with the vacuum grease sealing the hole created by the syringe's needle after the needle is withdrawn.

FIG. 15(b) is a diagram illustrating a top view of a case 155 that includes a resealable grease lock, in accordance with one embodiment of the invention. The case 155 includes a frame 158, a top cover and bottom (not shown). The frame 158 may be a gasket that is made from, without limitation, an acrylic foam or other suitable material that can be penetrated by a syringe or other dispenser. The frame 158 includes a hole 159 that is filled with grease or other self-sealing material, the hole 159 becoming enclosed when the frame is coupled to the top 157 and bottom to form the case 155. Fluid, such as immersion fluid 153 may then be dispensed through the frame 158 and grease-filled hole 159 using a syringe. Upon removal of the syringe, the self-sealing grease-filled hole 159 sufficiently seals the interior volume defined by the case 155. The resealable grease lock 156 may be in addition to a sealable opening on one side of the case 155 that can be used for inserting an array 152, as in above-described embodiments. Alternatively, the array 152 may be positioned within the interior volume of the case 155 during case 155 formation.

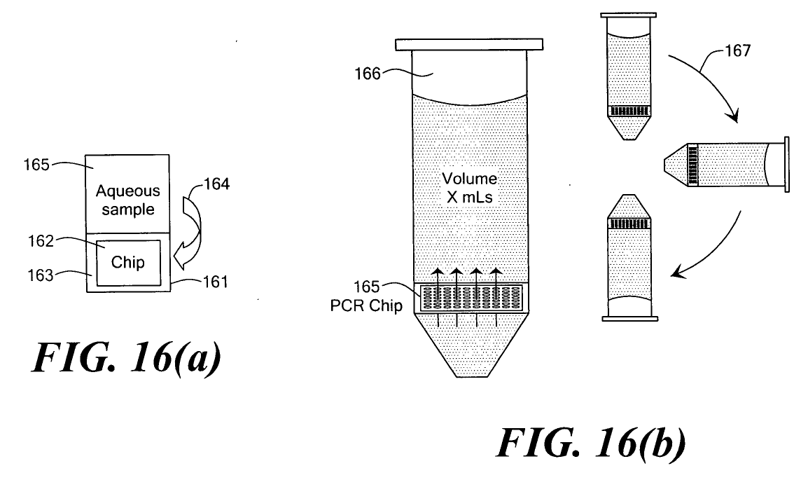

FIG. 16(a) is a diagram illustrating an embodiment of the present invention enabling the introduction of a sample into through-holes of a microfluidic array, in accordance with an embodiment of the present invention in which turbulence is introduced into the case. The array 162 may be sealed in a case 161 with both immersion fluid 163 and an aqueous sample 165, or aqueous sample alone. By causing the array 162 or sample to move back and forth, samples such as nucleic acids or proteins may be loaded into the chip. If a capture probe (described in more detail below) is included in through-holes of the array 162, the reciprocation will cause mixing of the sample and more rapid capture in through-holes of the array 162, which may be followed by an amplification such as PCR or ELISA. The fluid is desirably perfluorinated liquid and more dense than the sample, and thus the mixing, which may be done in combination with thermal cycling, is done preferably with the case in the vertical position with the array 161 at the bottom. The mixing may be effected by rocking, tumbling or spinning the case. The array 162 may be moved back and forth by other methods such as including magnetic materials in its construction (e.g. the array 162 itself or magnetic beads adhered) and dragging the array with a nearby magnet. The magnetic dragging mechanism may be integrated into a thermal cycler device. Structures may be placed on the array 162, such as beads or posts, which cause turbulent mixing to occur as the array 162 is dragged back and forth. This embodiment has the advantages of using a relatively low volume of liquid sample, reducing the number of steps necessary for loading/concentrating, being less error-prone in that a minimum of chip handling is done and convenience due to automation.

FIG. 16(b) is a diagram illustrating the introduction of a sample into through-holes of a microfluidic array by rotating the array, in accordance with an embodiment of the present invention. The array 165 is mounted in a tube 166. The tube 166 is then filled partly with sample and placed on a vertically oriented rotating disk (not shown). The rotation 167 of the disk forces the sample to flow repeatedly through the array 165, resulting in rapid sample concentration within the through-holes of the array 165. In other embodiments, the array 165 can be mounted to a bracket molded into the top of a screw cap, and then the cap can be attached to a plastic tube containing the sample to be analyzed. In still other embodiments, the array 165 may be sealed in a case with both immersion fluid and an aqueous sample 165, with the case attached to the rotating disk.

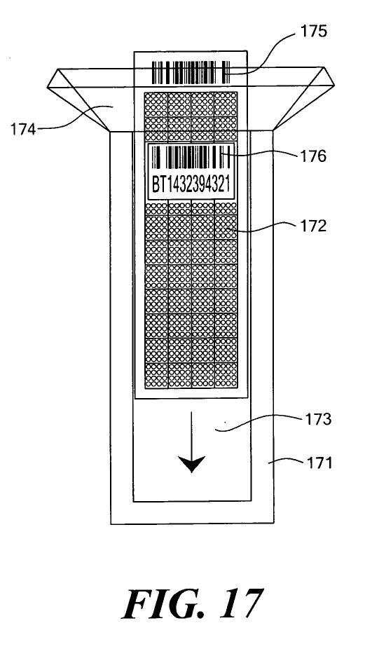

In further embodiments, a system and method for minimizing the volume of sample needed during loading of the array is provided. One limitation with the method described in FIG. 7 and FIG. 8 is that as the array 52 is lowered through the sample 72, the filling of the array 52 will reduce the volume of sample 72. If the total sample volume in the case 51 is lower than a critical value, the sample 72 will not remain as a horizontal layer as the array 52 passes through it, but will recede from the edges and assume the form of a droplet or droplets in or on top of the immiscible fluid. Thus, not all through-holes of the array may be populated with sample 72. Since the volume of sample 72 used must be enough to ensure that the total sample volume in the case 51 is higher than the critical value, this method may be costly in terms of the amount of sample 72 needed. Accordingly, various embodiments may advantageously ensure that the sample 72 remains spread in the form of a thin layer that extends across the width of the case 71 during the entire loading procedure. Such spreading means may be, for example, a region of hydrophilic material created on a background of hydrophobic material on the walls of the case 71. For example, the case 71 sides may be made from glass that has been silanized with OTS (octadecyl trichlorosilane) and then masked and exposed to a UV light to create hydrophilic stripes. These hydrophilic stripes may be rendered biocompatible by further treatment such as with a PEG-silane. In another embodiment, the spreading means may be in the form of a comb or brush, the sample retained in a stripe formed by fingers or bristles. FIG. 17 is a diagram illustrating an embodiment of the present invention facilitating the introduction of sample into through-holes of a microfluidic array 172, in accordance with an alternative embodiment of the present invention. In this embodiment, a funnel guide 174 is provided in contiguous relationship with the case 171. In this fashion, the introduction of sample material, in the manner discussed in connection with FIGS. 7 and 8 is facilitated and the minimum volume of sample needed is reduced. In various embodiments, the funnel guide 174 is integrated into the case 171. Alternatively, the funnel guide 174 may be a separate or removable item.

The funnel guide 174 may be of various shapes and sizes. For example, in one embodiment the funnel guide 174 may take the form of a trough with a narrow slit. The slit is of a narrow enough width such that sample will not pass through it when sample is placed in the funnel guide 174 above. The slit allows the array 172 to pass through it into the case 171 situated below. In a preferred embodiment, the slitted trough is made of a flexible material such as thin plastic that deforms to allow the array 172 to pass through the slit. The thin plastic provides slight contact and pressure against the array 172, preventing sample from leaking out of funnel guide 174 as well as facilitating sample loading in the array 172 and removal of excess sample on the array 172. As the user passes the array 172 through the sample and slit, the array 172 will fill with sample and pass into the case 171. If the case 171 is filled with immersion fluid 173 prior to insertion of the array 172, the amount of time that the filled array 172 is exposed to air and the amount of evaporation of the samples is advantageously minimized.

In order to further facilitate the entrainment of sample in the through-holes of the array 172, the funnel guide 174 may be provided with a series of fine brushes past which the through-holes of the array 172 move, with the result that, by capillary action, the sample in the funnel guide 174 is quickly guided into the through-holes. Note that the brushes may be used independently and/or regardless of the shape of the funnel 174, with the effect of spreading the sample out vertically and thus minimizing the amount of sample needed.

In FIG. 17, both the array 172 and case 171 are identified via barcodes 175 and 176, respectively. Other means of identification may be also be used as known in the art, such as printed labels that vary in color or shape, or smart labels having radio frequency transponders.

Thermal Cycling/Imaging/Analysis

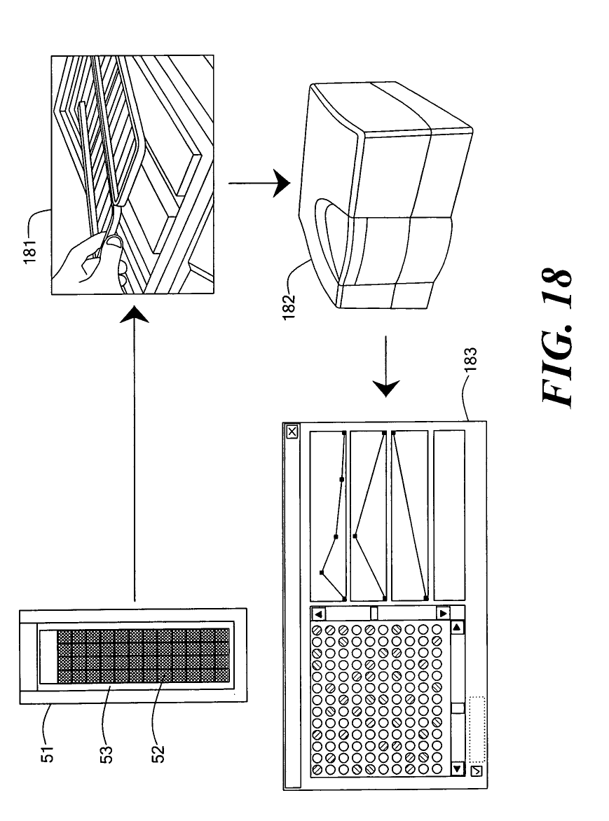

FIG. 18 is a diagram illustrating use of the sealed case of FIG. 15 in a thermal cycler 181, and in a scanner 182, so as to provide data that is subject to analysis using analysis software 183, in accordance with an embodiment of the present invention. In this fashion, the contents of each of the through-holes in the array may be cycled through alternating temperatures and subjected, for example, to analysis using Polymerase Chain Reaction (PCR) or Deoxyribonucleic Acid (DNA) sequencing techniques.

In various embodiments of the present invention, the thermal cycler 181 may be based, without limitation, on a temperature controlled circulating fluid or a temperature controlled thermal block. Both of these approaches are further described below.

Thermal Cycler With Circulating Fluid

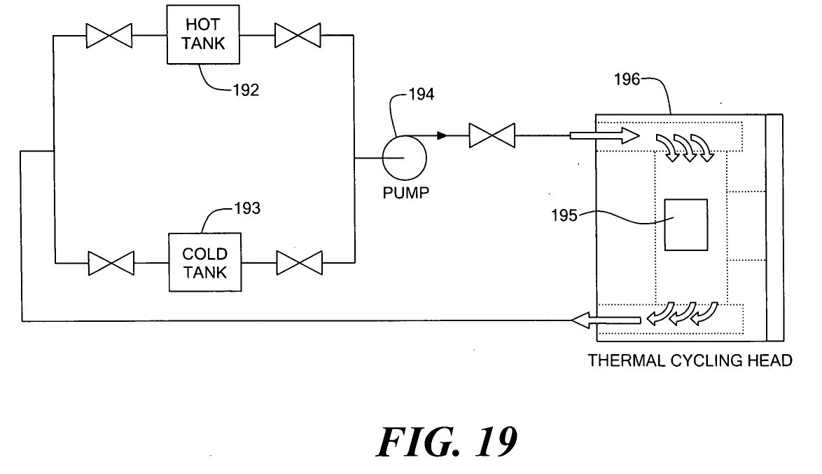

FIG. 19 is a diagram illustrating a high-density microfluidic thermal cycling system, in accordance with one embodiment of the invention. A case 195 containing an array, as described in above embodiments, is inserted into a thermal cycling head 191 that safely immerses the case 195 in a bath of controlled-temperature circulating fluid. A good circulating fluid possesses a high heat capacity, and specific examples include air, water and silicone oil. The cycling head 191 receives a circulating flow of fluid at a controlled temperature pumped from one of a hot tank 192 or a cold tank 193 by circulating pump 194. A valving arrangement allows for alternating selection between the two controlled-temperature storage tanks. Although FIG. 19 shows separate inlet and outlet valves for each tank, equivalent valving arrangements can be used, including valve manifold arrangements and multi-port valves, any of which may operated manually, pneumatically, or electrically.

The temperature of the fluid circulated through the cycling head 191 and past the case 195 is rapidly imparted to the array, allowing near-instantaneous temperature change to be uniformly applied to a large number of samples. For example, one embodiment processes 25,000 parallel PCR reactions simultaneously by producing 40 thermal cycles per hour.

The case 195 holding the array may be loaded by sliding it into a slot opening 196 in the cycling head 191, for example along a guide rail arrangement that holds the sealed case 195 in position in the flow of circulating fluid. Such an arrangement allows for vertical orientation of the case 191 and array (as shown, for example, in FIG. 15), which is not possible in prior art thermal cycling systems that are restricted to horizontal positioning of the array. Orientating the array vertically can be advantageous, for example, in preventing bubbles from getting stuck underneath the array, described in more detail below.

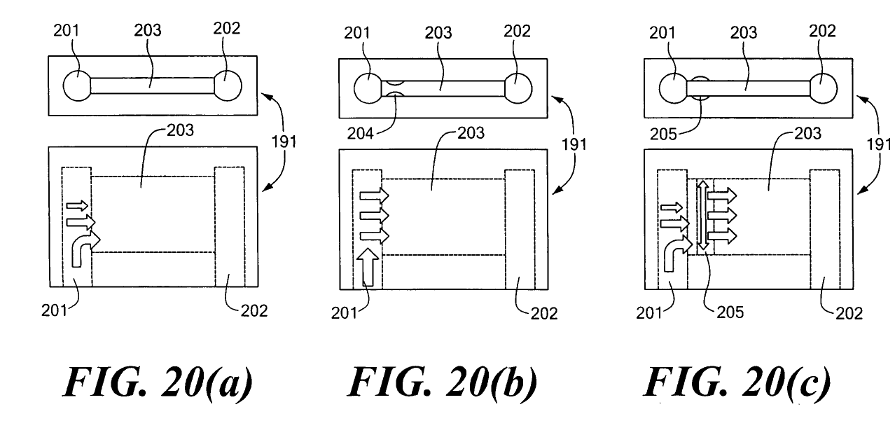

In some specific embodiments, the specific geometry of the cycling head 191 and specific mass flow rates of the circulating fluid could result in non-uniform fluid flow across the case 195. For example, as shown in FIG. 20(a), if the inlet port 201 and outlet port 202 of the thermal cycler 181 are smooth-bore cylindrical chambers, and if the connecting flow channel 203 has simple planar walls, the circulating fluid may flow preferentially across the portion of the case that is closest to the opening of the inlet port 201. This can be undesirable since it results in uneven temperature gradients across a case 195 that is inserted into the flow channel 203.

Such flow irregularities can be addressed by a flow regulator structure, which may be implemented in a variety of ways. FIG. 20(b) shows use of a flow restrictor 204 on the inlet side of the flow channel 203, towards the opening end of the inlet 201 to ensure even flow through the fluid channel. One variation of such a flow restrictor 204 utilizes one or more ridges added to the walls of the flow channel 203 to restrict the flow of fluid nearest to the opening of the inlet port 201. Such an arrangement minimizes eddies and dead zones in the flow, and promotes laminar flow of fluid in a uniform sheet over the case 195. This also helps create a more uniform temperature and to prevent bubbles from forming (which may distort sample imaging).

Alternatively, FIG. 20(c) shows a flow inlet cavity 205 upstream of the case 195 and on the inlet side of the flow channel 203 that acts as a flow regulator. The flow inlet cavity 205 may be wider than the case slot 196 and bounded by narrower regions on each side. This arrangement promotes fluid flow equalization across the case 195. Other flow control techniques can be implemented to address this issue, such as a straight-through flow arrangements.

With reference to FIG. 2, the top 22 and the bottom 23 of the case 195, which form the sides of the case 195 when the case 195 is in a vertical position, may be wholly or partly made of glass or other transparent material, and a corresponding section of the cycling head 191 may also be transparent. This allows for real-time imaging during thermal cycling, or convenient imaging before and after thermal cycling. Note that in other embodiments, imaging may be performed when the case 195 has been removed from, or may be independent of, the thermal cycling system.

Referring back to FIG. 19, other embodiments may have more or less than the two controlled-temperature storage tanks 192, 193. Alternatively, some assays may benefit from having three or more tanks at distinct controlled temperatures. Any arrangement of heating or cooling devices could be used to maintain the fluid in each tank at the desired controlled temperature. For example, heating coils and/or cooling coils may be immersed in any of the tanks.

Or there may be only one controlled-temperature storage tank, which is set at the lowest temperature (for example, in PCR or DNA sequencing, this would be the hybridization temperature, 55° C.). Higher temperature cycles could then be achieved by heating the circulating fluid prior to entry to the cycling head 191. For example, a heating coil could be wound around or embedded in a portion of the tubing between the outlet of the pump 194 and the cycling head 191. Instead of a heating coil arrangement, the circulating fluid could flow past one or more heated plates, such as a Peltier device, integrated into the cycling head 191 to heat the fluid. In any of these arrangements, a feedback loop could be used to precisely control the temperature of the circulating fluid.

In such an embodiment, it is advantageous to keep the temperature of the tank or tanks constant, so the fluid exiting the cycling head 191 should be cooled prior to its re-introduction to the tank or tanks. The circulating fluid could be cooled by a coil wound around or embedded in a portion of the tubing between the cycling head 191 and the controlled-temperature storage tank, or a cooling coil arrangement could be provided for the tank, again with a feedback loop to control temperature. Or, cooling plates, such as a Peltier device, could be integrated into the cycling head 191 to cool the circulating fluid as it exits the cycling head.

The advantages of a single tank system include faster heating times, more compact design, and less expense (fewer baths). Expense could be reduced even further by keeping the storage tank at room temp and actively controlling the temperature of the circulating fluid as it approaches the cycling head 191. A single controlled temperature environment could be useful on its own, for example, for drug screening.

In an embodiment having a temperature sensor, feedback control of the temperature signal could be used to automate the system. For example, automatic valve switching could be programmed to occur when a desired temperature is sensed. Such automatic and programmable operation is considered a customary feature of a thermal cycler. An embodiment may also feature automatic generation of melting-curve data by imaging as a function of temperature, e.g., after PCR with SYBR Green (Molecular Probes).

Thermal Cycler with Thermal Cycling Block

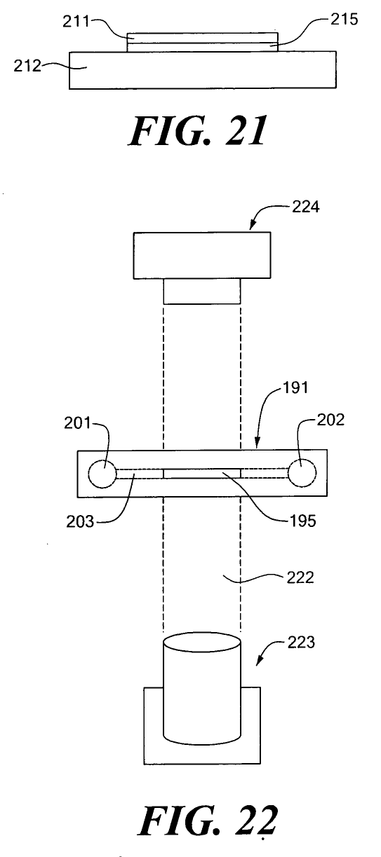

Instead of immersing the case 211 and/or array in a bath of controlled-temperature circulating fluid, the case 211 and/or array may be placed on a thermal cycling block such as a flat-block 212, as shown in FIG. 21, in accordance with one embodiment of the invention. The thermal cycling flat block 212 may be, without limitation, a thermoelectric device, such as a Peltier Effect cooling device, or other commercial available flat block thermal cycler, such as those sold by Applied Biosystems of Foster City, Calif. A Peltier Effect cooling device typically includes P-type and n-type semiconductor material connected electrically in series between two surfaces. When a voltage is applied to the semiconductor material, electrons pass from the p-type material to the n-type material, causing heat to be transferred from one surface to the other. The rate of heat transfer is proportional to the current and the number of p-n junctions.

A problem that occurs in thermal cycling reactions is that the temperature changes in the sample are often limited by the rate at which heat can leave or enter the Peltier device and be transferred to the samples. It is therefore advantageous to include one or more additional thermal contact means between the case and the thermal-cycling block. The thermal contact means may include a means for applying pressure to the case such as clips. Other embodiments that further increase heat transfer include use of a flexible heat transfer pad, grease, or paste. For example, a heat transfer pad 215, grease or paste may be placed between the flat block 212 (or the cycling head if a fluidic thermal cycler is used) and the case 211 holding the array. Flexible heat transfer pads 215, such as sold under the trade name Gap Pad (Bergquist Company, Chanhassen, Minn.), are typically thin sheets of elastomer containing material that enhances heat transfer. For example, the heat transfer pad 215 may be made of, without limitation, the following materials or combination of materials: silicone, graphite, fiberglass and/or assorted polymers. In various embodiments, the pad 215 may have an adhesive on one or both sides, or may be compressible such that pressure can be placed between the case 211, the heat transfer pad 215, and, for example, the thermal block 212, helping to ensure good thermal contact.

Rapid heat transfer is essential for optimal PCR biochemisty and throughput. The case preferably has a high thermal conductivity on the side, for example, that contacts the thermal cycling block and a low thermal mass to increase its responsiveness to changes in fluid flow temperature. The cycling head or flat plate may also have low thermal mass to ensure rapid thermal response time. Either the case, flat plate or the cycling head may include one or more temperature sensing devices such as a thermocouple probe.

The thermal cycling block may include a temperature control element, that may provide Proportional, Integral and Derivative (PID) temperature control, and that may include an offset designed to compensate between differences in heating rates of the thermal block and the array or arrays. The thermal cycling block may also include a slow ramp mode for melt curve analysis used for verifying the specificity of nucleic acid amplification reactions.

Additionally, the case may advantageously be made thin to increase the rate of heat transfer and reduce the amount of immiscible fluid needed. Note however, that if the case is too thin relative to the chip thickness, a gas bubble can form during thermal cycling and bridge from the chip surface to the case cover. This gas bubble causes condensation which can interfere with the PCR process and its imaging. Note however, that if the case is too thin relative to the chip thickness then the gap between chip and case may be small enough that a gas bubble that may form during thermal cycling can bridge from the chip surface to the case cover. This gas bubble could then cause evaporation and condensation which can interfere with the PCR process and its imaging.

Imaging

A transmission imaging system may be used where one side of the array, case and/or cycling head is illuminated with white light or other light source, and an imaging device (such as a CCD camera or scanner) on the other side receives a clear, well-illuminated image of the samples, in accordance with one embodiment of the invention. For example, as shown in FIG. 22, a transmission imaging system may be used where one side of the cycling head 191, or alternatively, just the case 225, is lit by a light beam 222 projected from a light source 223 at appropriate times or temperatures during thermal cycling. The light source 223 may be, without limitation, a white light source such as an arc light, and/or a laser scanning system. The sample through-holes in an array held by the case 225 are thus illuminated, and an imaging sensor 224 (such as a CCD camera) on the unlit side of the cycling head 191 receives a clear, well-illuminated image of the samples. In such a system, the material of the array may be reflective or opaque, e.g., silicon, and the imaging light does not reflect or bleed over into the imaging sensor 224. The illumination of the array may be off-axis from the camsera to minimize stray light entering the detector and may be from multiple angles as may be accomplished with the use of mirrors or fiber optic light guides.

In other embodiments of the invention, the imaging sensor 224 is on the same side as the illumination source 223, as for epi-flourescence imaging. A transparent array material—e.g. glass or plastic, or a opaque and dark material such as an array having black paint on the surface—is thus preferred to avoid reflections reaching the imaging sensor. An optical mask may also be incorporated into the case or imaging system to block light emanating from outside of the channels. In other embodiments, the array may include, for example, a reflective steel used in combination with angled illumination, as the angled illumination reduces reflections received by the camera.

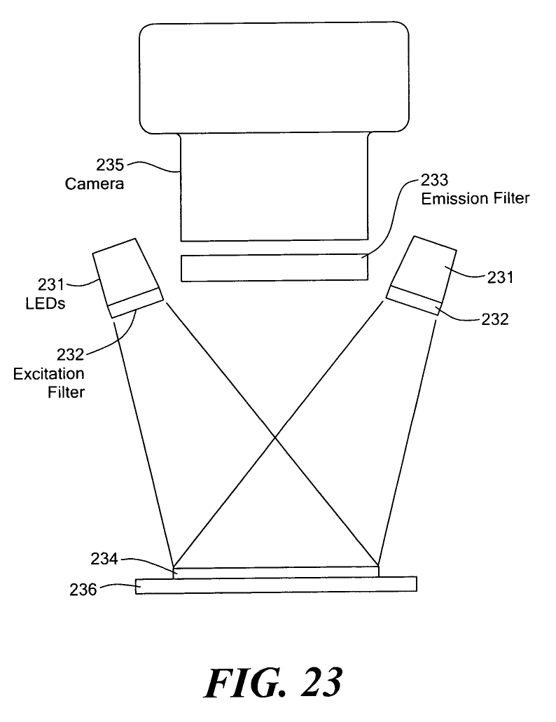

FIG. 23 is a diagram illustrating a epi-illumination imaging system for illuminating a microfluidic array 234 and the use of one or more Light Emitting Diodes (LEDs) 231 as an illumination source, rather than a white light source, in accordance with various embodiments of the invention. When white light is used, an excitation filter is used to choose the wavelengths that illuminate the sample, and the fluorescence is captured through an emission filter by a camera or other light sensitive device. Instead of a white light source, a bright LED or group of LED's 231 can be used in conjunction with an excitation filter 232. The LED's 231 are chosen by matching their central wavelength to the desired excitation wavelength; since much of the energy produced by the LED 231 is within the excitation spectrum, most of the LED light passes through the excitation filter 232. The sharpness of cutoff for the excitation filters 232 is less important than with white light since most of the light is in the excitation bandwidth, so cheaper filters 232 may be used. Additionally, if the spectrum of the LED 231 is narrow enough, the excitation filter 232 may be removed from the system altogether. Thus, the LED's 231 are more attractive than white light on account of their cost, size, efficiency, and simplicity.

The orientation of the array 234, which may be in a case situated on a thermal cycling flat plate 236 or contained within a cycling head, may be in any orientation with respect to gravity. In various embodiments, a symmetric set of LEDs 231 for each excitation wavelength to be imaged is placed off-axis from the camera 235. The symmetric positioning of the LEDs 231 is often advantageous to avoid shadowing in the three-dimensional through-holes of the array 236. Alternatively, a single set of LEDs may be positioned approximately on-axis that sufficiently illuminates a plurality, or all, of the through-holes of the array 236. Each set of LEDs 231 may include a plurality of LEDs. Alternatively, each set of LEDs 231 may include only a single LED having an output that is sufficient to illuminate a plurality of throughholes, such as, without limitation, a minimum output of 50 mW of radiometric power. The light from the LEDs 231 is columnated, with an angle of divergence from 0 deg to 90 deg. An excitation filter 232 is typically coupled to each LED source 231. The camera 235 is parallel to the surface of the case/array 236 (and/or cycling head 191), and an emission filter 233 is used on either side of the camera lens. A light shaping diffuser may be placed on the output of the LED's 231 to shape the light and provide better illumination uniformity.

The LEDs 231 may provide sufficient lighting to simultaneously illuminate the entire array 236, which may include, without limitation, from 100 to greater than 1600 through-holes and a through-hole density of, for example, greater than one through-hole per 0.25 mm². During fluorescence imaging for example, the fluorescence from each of the samples in each through-hole may then be simultaneously captured by the camera 235 as a digital image. The camera 235 may be, for example, a Charge-Coupled Device (CCD) or Complimentary Metal-oxide Semiconductor (CMOS) camera, which receives the image from each of the through-holes, or other sample site, simultaneously, and may, for example, transmit or otherwise process the digital image in serial format. The imaging lens of the camera 235 may advantageously be a MeVis lens, which may be directly mounted into the camera in place of the typical optical window and sealed tightly to prevent moisture and dust from entering the camera 235. In preferred embodiments, the camera 321 is of high enough resolution to discern individual features of the array. Intensity measurements for each sample can then be generated and the intensities processed by analysis software to generate desired data. In various embodiments, a plurality of replication cycles by Polymerase Chain Reaction (PCR) may be performed on the array 236 during thermal cycling, with the entire array 236 being simultaneously illuminated and imaged during each replication cycle.

Thermal Cycling System

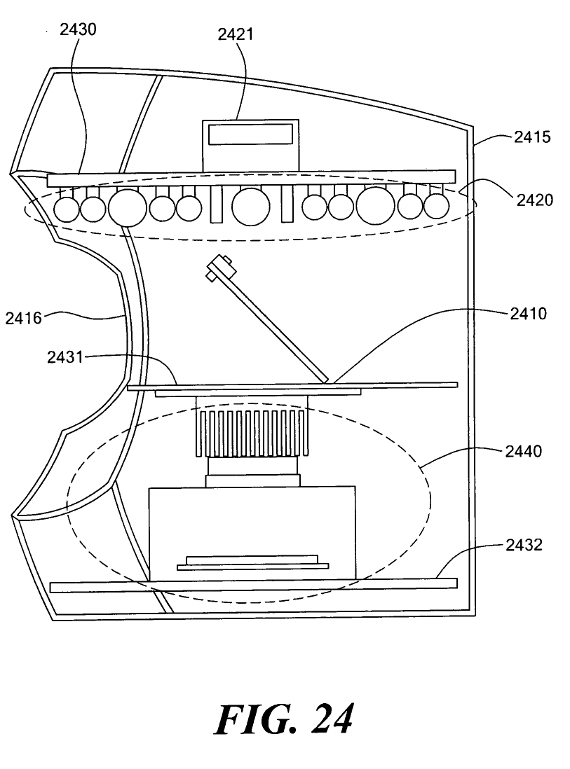

FIG. 24 is a cross-sectional side view of a thermal cycler system 300, in accordance with one embodiment of the invention. Using the thermal cycler system 300, contents of each of the through-holes in a microfluidic array may be, without limitation, cycled through alternating temperatures, imaged, and subjected, for example, to analysis using Polymerase Chain Reaction (PCR) or Deoxyribonucleic Acid (DNA) sequencing techniques.

The thermal cycler system 2410 may include, without limitation, a suitable enclosure 2415 having a hinged door 2416 for loading/accessing the microfluidic array, which in various embodiments is enclosed in a case, as described above. The enclosure 2416 may be advantageously light-tight when the door 2416 is closed, allowing for minimal background during imaging, such as when conducting fluorescence readings. The enclosure may include a sloped top oriented so that liquid spilled on the enclosure will safely run off and not enter, for example, an exhaust vent, which may be positioned on the side of the enclosure.

A thermal cycler 2410 is positioned within the enclosure 2415. The thermal cycler may include thermal cycling head with circulating fluid and/or a thermal block onto which the microfluidic array/case may be placed, each of which is described above.

In various embodiments, the thermal cycler 2410 is designed to provide heating and cooling as rapidly as possible, particularly when performing rapid and specific PCR reactions. With regard to a thermally cycler system that utilizeds a thermal block, liquid cooled Peltier devices are faster but are typically more expensive than air cooled Peltier devices. When using an air cooled Peltier device, a cooling fan may be used. The cooling fan may be advantageous remotely mounted from the thermal cycler and/or enclosure in order to minimize vibrations reaching the sample and/or optics, which may degrade imaging of the samples. This is contrast to conventional thermal cyclers which typically have a fan directly mounted on the surface acting as a heat sink, since vibrations are not typically an issue on the 96-well plate scale. A duct may be provided to direct flow of air from the remotely mounted cooling fan across the heat sink of the Peltier device. Air passing over the heat sink may be vented to the outside of the thermal cycler system through a grating.

A transmission imaging system is positioned within the enclosure 2415. As described above, the transmission imaging system includes various optics/light source 2420 for illuminating the microfluidic array, and an imaging device, described above. Imaging device may be a camera 2421, for simultaneously receiving a clear, well-illuminated image of a plurality of the samples, or a scanner which images each sample sequentially. The camera may be cooled. For example, the camera 2421 may be thermoelectrically cooled. One or more Light Emitting Diodes (LEDs) may be used as an illumination source 2420, rather than a white light or laser source, as described above.

Other components 2440 positioned within the enclosure 2415 may include, without limitation, one or more power supplies, circuit boards, heat sinks, and/or cooling ducts. As shown in the exemplary embodiment of FIG. 24, the optics/light source 2420 may be supported within the enclosure by a top plate 2430 located above thermal cycler 2410, which in turn, is positioned on a middle plate 2431. The other miscellaneous components 2440 may be positioned below the thermal cycler 2410 on bottom plate 2432.

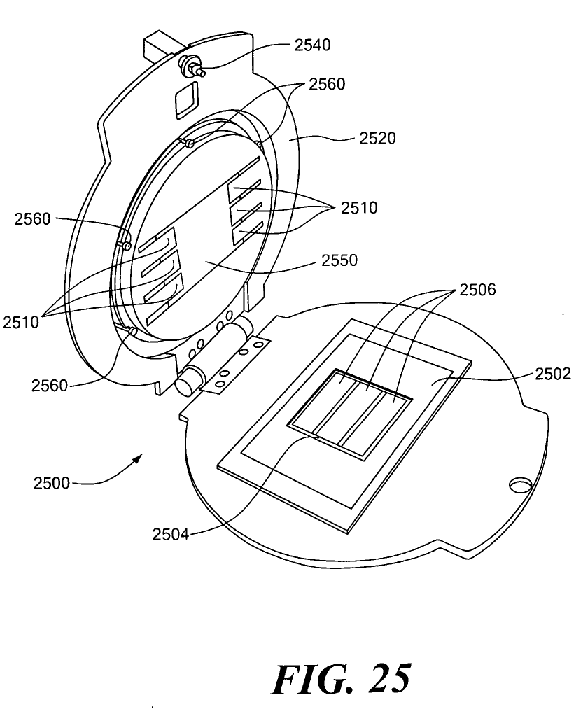

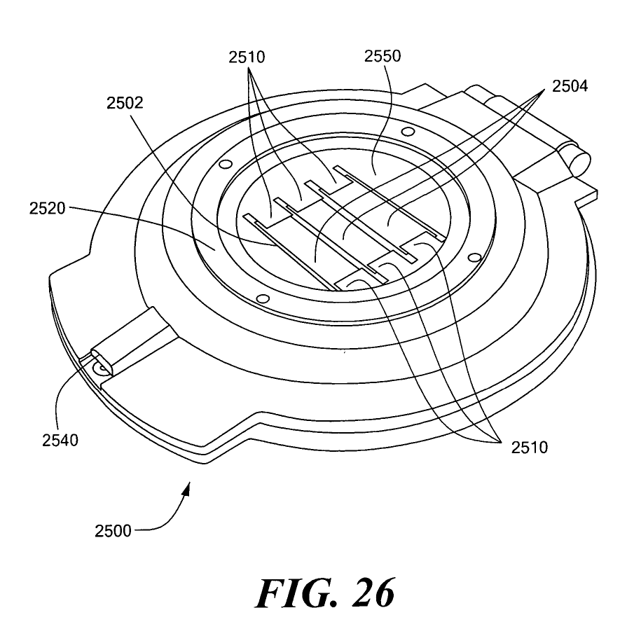

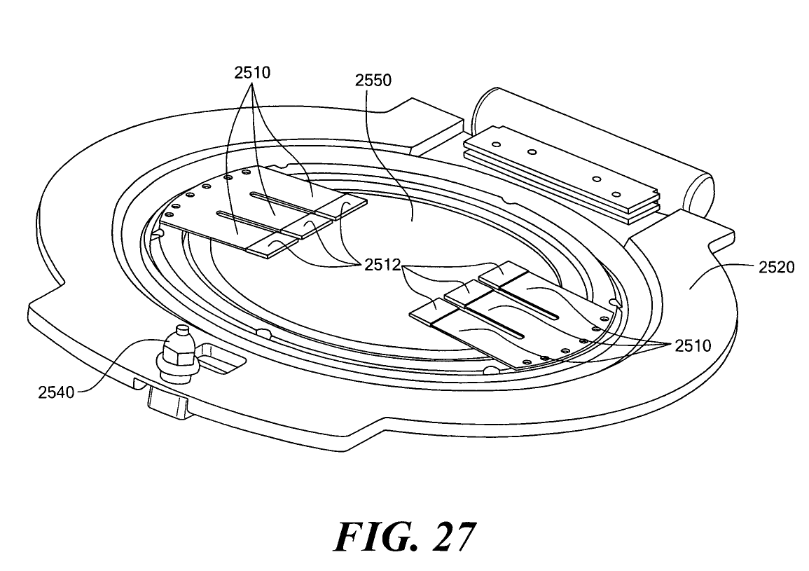

FIG. 25 is a perspective view of a thermal cycler with a lid assembly in the open position, in accordance with one embodiment of the invention; FIG. 26 is a perspective view of the thermal cycler of FIG. 25 with the lid assembly in the closed position; and FIG. 27 is a perspective view of a lid assembly, in accordance with one embodiment of the invention.

In illustrative embodiments of the invention, a positioning mechanism for easily positioning one or more microfluidic arrays/cases in a fixed position on a thermal block 2502 of a thermal cycler 2500 is provided. Fixing the position of the microfluidic array/case 2506 can be beneficial, for example, with regard to illumination and/or camera field of view. The positioning mechanism may be, without limitation, an indentation 2504 (shown in FIG. 25 with a microfluidic array case 2506 inserted) on the thermal block 2502 that position the microfluidic array/case 2506. When properly positioned, the microfluidic array/case 2506 rests in the indentation 2504. The indentation(s) 2504 may also advantageously serve to improve the rate and uniformity of heating a cooling the microfluidic array/case 2506, due, in part, to the additional metal contacting the sides of the case. Alternative positioning mechanisms may include, protrusions on the thermal plate, with the microfluidic array/case resting between the protrusions.

The thermal block 2502 may be polished smooth so that one or more cases 406 may be slid into the indentations 2504. The indentation 2504 may hold one or more cases 2506 and may further feature a graded portion such that microfluidic array/case 2506 may be slid into the indentation 2504 with a minimal of physical disturbance, which may cause loss of sample or cross-talk between retained samples.

In various embodiments, the thermal cycler/system 2500 may also feature a deck in close proximity to the thermal block, for placing the microfluidic array/case 2506 prior to loading and/or removal from the thermal block 2502. The surface of the deck 2502 is preferably smooth to facilitate sliding of the microfluidic array/case 2506. The deck 2502 may include an edge on which array-cases 2506 may be placed and then gently rotated onto the plane of the thermal block, thus preventing impact forces that may occur by dropping the array-case onto the surface and which may perturb the liquid samples. For example, the user may open the door to the thermal cycler/system 2500, scan a barcode (discussed in more detail below) on a rectangular 1 inch×3 inch case or a barcode on the array visible through the case, place the case 2506 on the deck and slide it down a ramp into an indentation that is approximately 3 inches×3 inches. This process may be repeated with additional array cases 2506 (dependent on the number of cases the thermal block holds) prior to closing of the door and initiation of thermal cycling.

As discussed above, rapid and uniform heating and cooling can be crucial to the throughput, reproducibility and general success of various reactions, such as PCR. Simply laying the array-cases on top of the thermal block 2502 often does not provide for optimally rapid and uniform temperature control of the array cases 2506.

In various embodiments of the invention, the thermal cycler/system 2500 may include a thermal transfer enhancement mechanism. The enhancement mechanism may be, without limitation, a mechanical element that presses the array-cases against the thermal block, thereby enhancing thermal transfer. For example, and with reference to FIGS. 25-27, each of the array cases 2506 may be pressed on with a set of fingers 2510 that may be positioned, for example, on at least one and preferably two or more edges of the array-case 2506. The fingers 2510 are preferably flexible and provide a defined and even amount of force across the area that they contact. The fingers may be made of, without limitation, a metal such as steel. The fingers may have an adequate footprint to limit the pressure created on the array-case and prevent bending of the case, which may cause contact between the case sides and the array and disturb the retained samples.

The heat capacity of the fingers, when making contact with the array case, may cause temperature non-uniformity across the array. To minimize the heat-wicking action of the fingers, the fingers may be made of, or coated with, a heat-insulating material 2512 such as rubber.

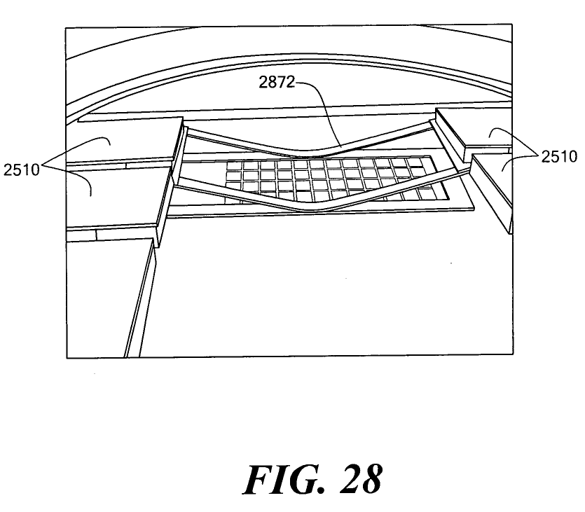

In various embodiments, in combination with, or in addition to fingers 2510, a spring device 2820 may be used to press down on the array case 2506, as shown in FIG. 28 in accordance with an embodiment of the invention. The spring device 2510 may, for example, span the fingers 2510 and contact the middle of the array case 2506, providing a force that presses the central region of the array-case against the thermal block.

In various embodiments of the invention, the thermal cycler/system 2500 includes a lid assembly 2520, which may be, for example, hinged to the thermal block 2502. The fingers 2510 (and/or spring device 2520) may be integrated into the hinged lid assembly 2520, such that the action of closing the lid assembly 2520 causes the fingers 2510 to contact the edges of the array-cases 2506, the array-cases 2506 preferably having been positioned by a positioning means such as one or more indentations 404 in the thermal block 2502. The fingers 2510 may be angled slightly downward so as to touch the cases 2506 before the lid assembly 2520 is fully closed, and generate pressure via bending action as the lid assembly 2520 is closed.

A gasket such as an elastomeric gasket, may be incorporated into the perimeter of the lid assembly 2520 in order to seal in heat and contain any evaporated encapsulating fluid or sample that may leak and possibly disadvantageously condense on the optical components of the system. The lid assembly 2520 may incorporate a closing-rate governor such as a friction hinge which allows the lid to remain open in any position, reducing the likelihood of disturbing the samples by closing at high velocity. Furthermore, the lid assembly may incorporate a latch 440 to hold it closed. The latch 2540 may preferably allow for one handed operation and provide a mechanical advantage for conveniently compressing the elastomeric gasket.

In various embodiments, the lid assembly 2520 may feature a stop that prevents it from fully opening. For example, the lid may only open to about 45 degrees so as to prevent it from contacting closely positioned optical components. Such a design allows for a more compact and light-efficient design.

To prevent the lid assembly 2520 from heating up over multiple thermal cycles and raising the average temperature of the arrays over time, the lid or array-case 406 temperature may be measured. A control element, which may include, without limitation, a microprocessor and associated software, may then adjust the heating and cooling time or power as a function of the temperature measurements. In various embodiments, the lid assembly 2520 may be made of an insulating and/or low thermal mass material such as plastic that may be reinforced with steel rings. The lid assembly 2520 may be advantageously placed on a platform that may be adjusted for its angle and height relative to the rest of the imaging system, such as the camera and illumination optics.

In order to perform imaging of the samples such as during real-time thermal cycling, the lid assembly 2520 may further comprise a transparent region or optical window 2550. The window 2550 may be advantageously large to allow visualization of all of the retained array samples. Positive stops such as one or more posts 460 of a defined length may be incorporated into the lid or thermal block to maintain a uniform and defined distance between the optical window and the thermal block to improve imaging flatness. To improve the imaging of the arrays and reduce the footprint of the apparatus, the transparent window 2550 may include a lens. The lens may include a full-field plano-concave lens to provide for a flatter image both by directing excitation light across the array more evenly and providing to the camera a flatter fluorescence emission image.

A potential problem when using partially volatile immersion fluids such as perfluorinated liquids is that should a leak develop in the case, such liquids may evaporate at high temperature and condense on optical components—causing a fog that interferes with data collection and analysis. A defogging mechanism may thus be provided, in accordance with an embodiment of the invention. The defogging mechanism may include, without limitation, a heating element for heating the optical components, and a cold surface element for condensing liquids. The heating element may include electrical heating elements or/and an infrared lamps. The cold surface element may include a thermoelectrically cooled surface. Another embodiment is to coat the optical components with an optically transparent hydrophilic layer to prevent the condensate from froming droplets on the optical surfaces, thereby ensuring the integrity of the imaging path despite the presence of a condensed liquid on the optical surfaces. This assumes, of course, the liquid does not in and of itself interfere with the array illumination and imaging.

The thermal cycling system may include a microprocessor and associated software that provides temperature control, illumination control, data collection and analysis. When imaging miniaturized reactions, a spot-finding and integration algorithm may be used to convert raw images into spot intensities, as known in the bio-array art. For real-time PCR applications, data analysis typically involves the setting of a threshold value, such that the cycle number at which the relative fluorescence intensity of the sample crosses this threshold is correlated with the initial concentration of target nucleic acid. An algorithm may be used for setting this threshold value that includes selecting multiple trial threshold values and determining which trial value produces the best fit to a standard curve produced from samples of known target concentration. A large number of trial threshold values may be used or an automatic optimization approach may be used.

Orientation of the case when thermal cycling can be a factor when thermal cycling. Although horizontal or hybrid orientation of the array is acceptable for many embodiments, vertical orientation of the case 195 advantageously allows bubbles that form in the immiscible fluid in the case 195 to float up rather than getting stuck underneath the array. Such bubbles could distort imaging of the samples, and also can lead to evaporation of the samples within the array, even through perfluorinated liquid. In various embodiments, thermal cycling in a vertical position can be performed before sealing of the case 195 to allow any gas bubbles or vapor that may be a generated to escape before sealing. This contrasts with a horizontal orientation structure, in which an inlet and outlet tube arrangement would be typically used in order to fill the case 195 completely with immiscible fluid, without leaving any air. In alternative embodiments, thermal cycling in the vertical can be performed without sealing of the case since the contents will not spill in this orientation.

Other techniques, with the case 195 in a vertical, horizontal, or hybrid orientation, may also be used to reduce the formation of undesirable bubble formation. For example, the case 195 may be made rigid, such that the case 195 does not expand due to increased temperatures during thermal cycling. Since the volume within the case 195 is held constant, the pressure increases, preventing formation of undesirable bubbles.

In various embodiments, a salt, or other osmolyte, may be added to the sample or other fluids contained within the case. Since the boiling point is elevated by the osmolyte, outgassing of air in the aqueous sample is reduced, along with evaporation of water. The salt may be added, without limitation, to the sample before dipping of the array, or may be introduced during encapsulation. Small molecule osmolytes such as sugars, including glycerol, are generally suitable. Other osmolytes or hydrophilic polymers that do not interfere with the desired reaction can also be used. For example, PEG, polyvinyl pyrrolidone, polyvinyl alcohol, polyacrylates, KCl, NaCl, or Tris buffers may be used. Amino acids, such as glycine, in the range of 0.1M to 3M, but more preferably between 0.2M and 2M, are also suitable. Betaine (an amino acid) at up to about 2M may be used to prevent evaporation and improve PCR reactions on target sequences rich in G-C (as opposed to A-T).

In various embodiments of the invention, an immersion fluid is provided that does not outgas, especially during thermal cycling. The property of not outgassing may be important to prevent bubbles from forming in the immersion fluid during thermal cycling and interfering with data collection.

In accordance with one embodiment of the invention, removing dissolved gases from the fluid(s) may be accomplished by exposing the liquid to a vacuum at a pressure lower than ambient pressure. Any dissolved gas migrates to the surface and exits, thus effectively decreasing the amount of gas dissolved in the liquid with time and with increased pressure difference. The maximum pressure difference applied to the liquid should not exceed the fluid vapor pressure to avoid excessive evaporation of the immersion liquid during degassing.

In other embodiments of the invention, the immersion fluid may be heated to the fluid boiling point to remove dissolve gas in the fluid. The time at the boiling temperature is limited to prevent excessive evaporation of the liquid. Still other embodiments of the invention may include combining reduced pressure and increased temperature to degas the liquid.

Another method of removing dissolved gases from the fluid(s) is by sparging with helium, then removing the gas by evacuation. During sparging, a stream of helium bubbles, for example, is passed through the immersion fluid so as to sweep dissolved air out of the fluid liquids, thereby limiting the formation of air bubbles during thermally cycling. The helium remains soluble at all the temperatures used in the thermal cycler and so does not create bubbles itself. Perfluorinated alkane liquids (such as Fluorinert™ FC-70 from 3M) prepared in accordance with this method may advantageously not only not outgas, but tend to absorb gasses released from the aqueous samples in the microfluidic array and thus prevent bubbles from forming in the encapsulant fluid or in the retained sample. For convenience, vials of pre-degassed liquid may be provided that can be immediately opened and used. To produce the pre-degassed liquid, the fluid may be sparged with a sparging gas, and then packaged in a container that retains the fluid and selectively keeps out air, but allows the sparging gas to escape. For example, the container may be a plastic vial for holding, without limitation, about 1 mL of perfluorinated alkane liquid sparged with helium; after packaging, the helium will leak out leaving a degassed liquid.

The thermal cycler system may include a barcode scanner that is operatively connected to either an internal or attached computer. The barcode scanner may be positioned, for example, on the deck to the thermal block (described above). In various embodiments, the barcode scanner may be capable of, without limitation, reading a barcode on the case, or on the array through the case (allowing for case interchangeability).

Polymerase Chain Reaction

In a further embodiment, Polymerase Chain Reaction (PCR) can be performed using very small amounts of genetic material. During PCR, a series of heating and cooling cycles via a thermal cycler is used to replicate a small amount of DNA. Through the use of various probes and/or dyes, the method can be used analytically to determine the presence or amount of a particular nucleic acid sequence present in a sample.

In a specific embodiment, reagents such as primers or fluorescence probes may be immobilized in the through-holes by encapsulation in a wax. This wax is preferably hydrophilic and biocompatible so that it dissolves and releases the reagents upon heating. For example, an array of immobilized primers and TaqMan probes comprising thousands of genotyping or RNA expression assays may be created by encapsulating the primers and probes in polyethylene glycol (PEG) on the walls of the through-holes. The sample containing the nucleic acids to be analyzed is then introduced and the array is thermal cycled with real-time analysis which may be accomplished by the instrumentation described herein.

For genotyping applications, the assay described in U.S. provisional patent application No. 60/528,461, entitled “Improved Selective Ligation and Amplification Assay” filed Dec. 10, 2003, which has been incorporated by reference in its entirety, provides an advantageous assay system in that many specific and inexpensive assays may be quickly designed. The assay allows for identifying and distinguishing a nucleotide polymorphism in a target sequence of nucleic acid in each through-hole of the array. The assay includes three or more primers, two of which bind to a target nucleic acid sequence, flanking a SNP, so that the 3′-end of one or more first primers is adjacent to the 5′-end of a second primer, the two primers being selectively ligated and then amplified by a third primer to exponentially produce the complementary strand of the target sequence. The other strand of the target sequences is exponentially amplified by un-ligated first primer. Using a microfluid array, an SNP in a target sequence of nucleic acid can be thus be advantageously identified. In various embodiments, a kit may be provided that includes the microfluidic array chip, primer sequences, and reagents required to selectively ligate primers for amplification of a desired target nucleic acid sequence.

Alternatively, the encapsulated components could be an array of samples for probing with one or a few assays; for example, immobilized patient DNA samples for use in epidemiological studies. In some cases, the entire array could have the sample immobilized assay system which may be used, for example, in haplotyping by limiting dilution PCR. For some applications it may be desirable to combine both genotyping and RNA expression analysis assays in the same array which may be advantageous for sample tracking as in for patient samples.

It is important to note that simply drying the reagents onto the walls of the through-holes without an encapsulating matrix would be problematic in that if the sample is loaded by dipping of the array, dragging of droplets across the array, or other method that exposed the sample to multiple through-holes simultaneously, the reagents may dissolve and contaminate neighboring channels as well as reduce the reliability of results in the channels that lost material. This is of especially high importance is target molecules are array as for studies of patient populations since target molecules are amplified by PCR whereas primers and probes are not. A means for reducing this crosstalk may be implemented in the array such as adding a second layer of protective wax. The composition of this second layer may be the same as for the first layer, or may differ.

For many assays, it is important that the interior surfaces of the through-holes (the walls) are biocompatible so that they do not interfere with the reaction by adsorbing, denaturing, reacting with or catalytically destroying the assay components. For this reason, it is preferable to coat the walls with a biocompatible material. This material could be for example, a covalently linked PEG bearing silane. This coating should be thermally stable at the highest temperatures used in the assay (typically 95° C. for PCR).

In order to increase the sensitivity of the assay a sequence capture-PCR array may be created. The through-holes of an array 2872, such as the one shown in FIG. 28, may be provided with an array of sequence specific hybridization capture probes, in accordance with one embodiment of the invention. The probes may be, without limitation, immobilized on the interior walls of the throughholes of the array 2872, or on a porous material embedded within the throughholes. A sample containing a nucleic acid to be amplified is allowed to hybridize to the probes as is common for hybridization arrays. The array 2872 may be washed in a buffer designed to remove non-specifically bound nucleic acids. PCR reagents are then introduced into the sample array 2872 by stacking with a second through-hole array or by other means. For example, the second array may contain primers that specifically amplify the sequence complementary to the probes, or may contain universal primers. Thermal cycling and analysis can then be performed. More detail on adapting the through-holes of the array 2872 for functional processing of a sample, and stacking of arrays 2872, is provided in the section below.

In one specific embodiment, the array 2872 may include at least three different reagent oligonucleotides: (1) a capture probe oligo immobilized on the through-hole wall having a high specificity for the target DNA, and (2) a forward PCR primer and (3) a reverse PCR primer for amplification of the target DNA. Such an approach provides high specificity for the target DNA based on three different domains of specificity that must be met.

The advantages of such embodiments include a reduction of template sample mass requirements by greater than 10-fold (greater than 100-fold in some embodiments), and increased specificity of the output by combining specific hybridization with the specificity inherent in the PCR sequencing. Similar embodiments are also compatible with techniques other than PCR, such as DNA sequencing or non-thermal amplification systems.

Single and Multi-functional Assays

In illustrative embodiments of the invention, individual through-holes of the sample array are adapted for single or multi-functional processing of a liquid sample. Single or multi-functional processing may include the capture of one or more targets of interest and/or chemical processing of the captured targets. The target capture may be based on a nucleic acid probe, protein antibody, aptamer or other capture agent of material immobilized within the through-holes. The chemical processing may use immobilized reagents that serve to modify the captured targets.

In one embodiment, the chemical processing includes amplifying and detecting a signal from the captured targets. For example, the chemical processing may utilize encapsulated TaqMan® PCR reagents, or reagents for some other nucleic acid detection scheme. In some embodiments, the chemical processing may be specific to the captured targets. For example, the target capture can use oligonucleotides immobilized within the through-holes to specifically capture target nucleic acids in a sample, such as by a stringent hybridization. The chemical processing then may use TaqMan® reagents with primers and probes specific to the target nucleic acids captured by the immobilized oligonucleotides.

The assay reagents such as primers, molecular probes, proteins, antibodies, enzymes, enzyme-antibody conjugates, nucleotides, oligonucleotides, fluorimetric substrates, buffers, salts, blocking agents, or some other assay component can be immobilized within the through-holes in a variety of manners so as to release the substances upon activation into aqueous solution within the sample through-hole. Activation may be triggered, for example, via prolonged incubation or by exposure to heat, light, solvent, pH, oxidant, reducing agent, or some other trigger. These immobilization techniques include covalent attachment, non-covalent attachment, and immobilization in a material with good surface adherence properties such as polyethylene glycol (PEG). Hereinafter such materials will be referred to as waxes. Preferentially, the wax should be hydrophilic to facilitate loading of the through-holes by use of surface energy. The wax should also be biocompatible so as not to interfere with the reaction or detection system. In some applications, the chip may be exposed to elevated temperatures (e.g., around 40° C.) for several hours, and thus the wax may need to have a higher melting point (or be sealed-in with a layer of high-melting wax).

Assay reagents such as probes and primers may be mixed with wax and transferred from reagent stocks in microplates into the sample through-holes in the multi-functional chip, for example by use of a high-accuracy robotic pin tool. The prepared chips are then dried to immobilize reagents such as PCR primers and probes on the walls of the sample through-holes. If the wax is hydrophilic, a solution containing a target of interest such as a patient's DNA and a polymerase (such as Taq) along with other reagents needed for PCR can be loaded into the through-holes by dipping or other means, as described above. Upon thermal cycling, the wax will melt and dissolve, releasing the nucleic acid component.

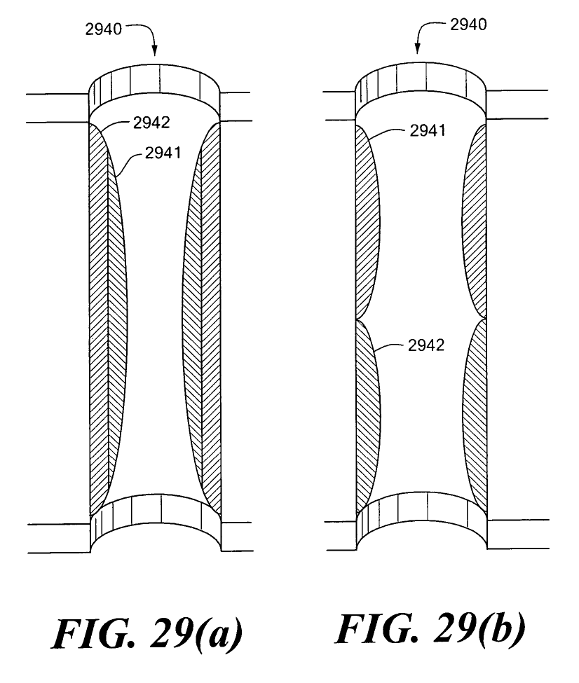

In some embodiments, multiple reagents are dried in multiple layers of wax within the through-holes. FIG. 29(a) shows a through-hole 2940 having an outer first layer of wax 2941 displaying target capture reagents, and an inner second layer of wax 2942 having chemical process reagents. FIG. 29(b) shows an alternative embodiment in which the first layer of wax 2941 and the second layer of wax 2942 are attached to the interior walls of the through-hole 2940 at different locations. In either embodiment, each layer of wax may have different melting temperatures (e.g., different polymer lengths) to allow sequential activation of these reagents at different temperatures. In FIG. 29(a), this would mean that outer first layer of wax 2941 would have a lower melting point than the inner second layer of wax 2942. This can be easily accomplished simply by applying and drying the lower melting point wax after the higher melting point one.

In some embodiments, the double layer wax structure may be present in only a selected subset of the through-holes in order to enable multiple types of analysis such as RNA and DNA analysis or ELISA and PCR analysis on the same chip. In other words, the immobilized reagents can vary from through-hole to through-hole to provide multiple types of information (e.g., SNP, gene expression patterns, etc.) on one or more samples.

Such a layered wax chip is useful, for example, for a two-step reverse transcription/PCR system in which the reverse transcription copies sample RNA to DNA, and then PCR processing amplifies the DNA as for detection, such as by Quantitative PCR(QPCR)). The required PCR primers and probes are dried down in the sample through-holes first in wax that melts at 65° C. Then primers for the reverse transcription reaction are dried over the first wax layer in a second top layer of wax that melts at 45° C.

The RNA sample (such as from an RNA virus) along with a one-tube RT-PCR master mix with a thermostable reverse transcriptase (available, for example, as SuperScript™ from Invitrogen Corporation of Carlsbad, Calif.) can then be added and heated up to 50° C. to release the reverse transcription primers and then incubated at 37° C. to allow the reverse transcriptase reaction to occur. The maximum temperature used in various applications can vary within the temperature stability limits of the enzyme. Then the chip is thermally cycled to release the PCR primers and probes and perform the PCR amplification and analysis. An additional level of specificity may be gained in the assay by using different probes for the RT and corresponding PCR. This technique can also be used in other sorts of assays where time or temperature sequential addition of reagents is required.

Layers of multiple melting point waxes may also be useful for reducing sample cross-talk (cross-contamination) that might result from immobilized nucleic acids traveling to nearby through-holes, such as during the sample dipping/loading process. This may involve an outer protective layer of wax that shields the lower layer(s) of wax. This protective layer of wax could be the same or different composition as the underlying layer(s).

Layered wax embodiments provide great design flexibility. For example, the target capture process need not have nucleic acid probes, but could be used to isolate viral particles directly as by affinity capture with immobilized antibodies. The chip is then washed and the nucleic acids are released by heat, lytic enzymes, or other means. If further purification, specificity, or nucleic acid stability is needed, oligo-capture probes may be mixed with the antibody capture probes. In this case, an on-chip reverse transcription reaction is necessary. Lytic enzymes may be chosen to denature upon heating and thus not affect the reverse transcriptase or polymerase needed for PCR.

In various embodiments, multiple functionalities may be integrated into a multifunctional chip by producing multiple chips containing complementary reagents. Then, two (or more) chips can be layered together to form a single integrated multi-functional chip. Some embodiments may start by bonding separate dedicated capture and chemical processing chips such that the chemical processing functionalities in the through-holes of the chemical processing chip will align with the appropriate capture functionalities in the capture chip. In some embodiments, it may be possible to mix the capture and chemical processing functionalities between the two chips as long as the correspondence between the capture and chemical processing functionalities is maintained.

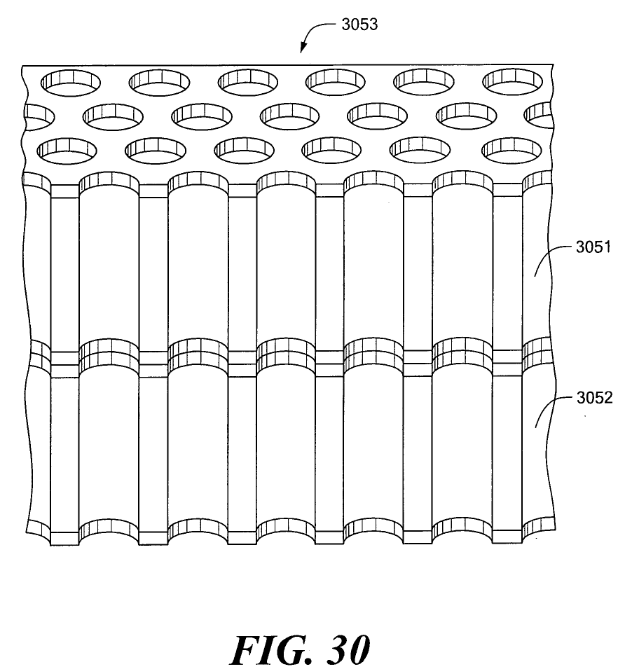

FIG. 30 shows an embodiment in which a top chip layer 3051 is stacked directly onto a bottom chip layer 3052. Although FIG. 30 shows two different chip layers, other embodiments could have three or more chip layers. The chip layers are aligned so that the through-holes in each are aligned together, and the two chip layers 3051 and 3052 are fixedly connected to each other to form a single unified layered structure 3053. Multiple chip layers 3051 and 3052 can be attached to each other in various apparent ways such as by use of adhesives, chemical cross linkers, screwing, bolting, riveting, clamping, etc. Or if the surfaces of the chip layers 3051 and 3052 are polished or sufficiently flat, they may be bonded directly using pressure or by use of Van Der Waals forces.

Many different nucleic acid component sets such as sets of hybridization probes and PCR primers can be preloaded into the layered chip in this way for rapid analysis. The loading of the nucleic acid component or samples to be analyzed may be accomplished in various ways such as by pipetting a solution containing the nucleic acid component directly into the sample through-holes, or by dragging a drop of solution containing the nucleic acid component over the openings of the sample through-holes. Or, the chip layer can be dipped in a solution containing the nucleic acid component, and then withdrawn. Alternatively, arrays of nucleic acid targets as might be obtained from numerous patient samples may be immobilized and then loaded with reagents such as PCR master-mix containing primers and probes. Once a total number of DNA detection assays is established for a given specific application, the number of through-holes may be reduced to minimize non-specific binding by the unused through-holes. The openings of unused through-holes may be blocked with wax to prevent non-specific binding of the sample target DNA.

For example, such a layered chip may provide DNA capture and amplification in which one chip layer captures DNA of interest in a liquid sample onto an array of oligonucleotides covalently linked to the hydrophilic surfaces of the through-holes, while another chip layer amplifies the captured DNA such as by PCR.

The PCR primers and probes encapsulated in the array of through-holes of the second chip layer may be specific for the targets captured by the oligonucleotides in those through-holes. In an example diagnostic assay, this enables multiple assays per pathogen against numerous pathogens and replicate analyses to increase data quality. The flow-through nature of such a multi-functional chip may be used to facilitate target concentration, purification, and amplification, which increases nucleic acid detection sensitivity by as much as an order of magnitude or more compared to previous nucleic acid analysis methods. Some embodiments could have a combination of multiple chip layers as well as one or more layers of reagent-bearing wax such as described above.

In a DNA capture and amplification embodiment, the capture chip layer has specific nucleic acid probes (e.g. 40-60 mers of DNA) attached to the sides of the sample through-holes. Robust interior oligonucleotide-capture surface coatings may be used consistent with the goal of minimizing non-specific binding. Established chemistries for immobilizing oligonucleotides onto surfaces may be exploited. For example, oxide surfaces (such as glass) may be modified with undecenyltrichlorosilane to produce a monolayer exposing a vinyl group carboxylate at its end, which is functionalized to carboxylic acid by exposing to KMnO4/NaIO4 in aqueous solution. The carboxylic acid is activated to NHS ester by subsequent exposure to 1-Ethyl-3-(3-dimethylanomipropyl) carbodiimide hydrochloride (EDC) and N-hydroxysuccinimide (NHS) ester. Oligonucleotides or cDNA strands bearing an amine group at its end could then be immobilized to the surfaces by forming amide bonds via the reaction between NHS ester on the surface and amine group in the strands. The amide bond and underlying undecenyltrichlorosilane monolayer are expected to provide sufficiently robust linkage to retain the strands on the surface under hybridization conditions.

The different chip layers should be mechanically bound together in precision alignment so that the through-holes containing complementary PCR primers and hybridization probes in each layer are aligned. A hermetical bond may be desirable but is not necessarily needed provided that the chip layer surfaces in contact are hydrophobically coated. In this case, the layer bonding process also should not modify the coating hydrophobicity to ensure fluidic isolation between adjacent through-holes. In one specific embodiment, the two chip layer exterior faces are pre-coated with reactive monolayers prior to filling with assay probes, then bonded together by catalyst-activated crosslinking.

If adhesives are applied after the probes are added, or after the hybridization step, then the adhesive application process should minimize spillover into the through-holes since adhesives may inhibit PCR or bind target oligos. Excess adhesives may be washed away from the through-hole interiors with solvents that do not dissolve the encapsulating wax. The bonding process should also work near room temperature so as not to melt any probe-encapsulation wax, and should ideally be done in a manner that does not contaminate the chip with dirt or nucleic acid contaminants (though washing is possible). This may require testing of different pressure sensitive adhesives and dispensing mechanisms such as sprayers, rollers and stamps to develop a means of applying uniform pressure. Alignment can be accomplished by the use of a precision jig having pins complementary to guide holes that are precision etched during the chip layer manufacturing process. If needed, chips can be blocked with a blocking agent such as bovine serum albumin (BSA) to occupy any binding sites created in the bonding process. Hybridization buffers and PCR master mix may be formulated with dynamic blockers to improve their compatibility with the adhesive layer.

The capture chip layer works in a manner similar to a standard glass-slide spotted hybridization array - nucleic acids may be diluted in a buffer designed to optimize speed and/or specificity of hybridization and have a chance to visit all of the sample through-holes of the capture chip layer and thus come to a low free-energy state of complementary hybridization. Alternatively, the hybridization may occur in a crude or diluted patient sample such as a nasopharyngeal wash sample. Enzyme may be used to disrupt pathogens prior to hybridization.

The capture chip layer may be incubated with a nucleic acid sample for 6 hours or more as with a standard microarray. This incubation time may be reduced by circulating sample through and around the chips, but the wax encapsulation matrix encasing the PCR primers and probes needs to resist dissolution until the thermal cycling is initiated by heating to 95° C. Additionally, stringency can be controlled by lowering salt concentrations, resulting in lower incubating temperatures. In some applications there may be two additional options: (1) decrease the hybridization temperatures and sacrifice specificity of hybridization and possibly limit detection, or (2) manually stack the chip with amplification reagents onto the capture chip after the hybridization step. Manual stacking methods have been described in U.S. patent application Ser. No. 09/850,123, entitled “Methods for Screening Substances in a Microwell Array,” filed May 7, 2001, which is herein incorporated by reference. Manual stacking may involve, for example, the steps of stacking at least two platens together in such an adjacent manner that at least one of the plurality of through-holes from each platen is registered with a through-hole of each other adjacent platen so as to form at least one continuous channel, and transfering the liquid into each continuous channel. Each platen may be separated from each adjacent platen by an air gap, and the liquid may be transferred with capillary tubes or at least one cannula.

Hybridization reaction kinetics are diffusion-rate limited and given that the diffusion constant for nucleic acids is small (˜10⁻⁶ cm²/s), diffusion into or within the through-holes may not be enough for rapid hybridization. This problem may be addressed by increasing the surface capture area within each through-hole such as by actively circulating sample to repetitively force it through the capture chip layer. Surface capture area can also be increased by introduction of a porous matrix into each through-hole that can be functionalized with hybridization capture probes. Matrix porosity should be selected to maximize surface area while minimizing the pressure required for liquid flow through the through-holes. For example, porous glass may be synthesized in the through-holes by filling the through-holes with a mixture of potassium silicate mixed with formamide, and then baking at 110° C. for one hour. By varying the concentration of formamide or including particles such as porous silica or polymer beads in the potassium silicate mix, the porosity of the matrix can be adjusted as desired. Furthermore, immobilization chemistry as described herein can be used to attach capture probes to the glass surface. In other embodiments, alternatives such as polyacrylamide, agar or aero gels can be used.

To increase hybridization rates, the chip can be spun/rotated (see, for example, FIGS. 167(a-b). Alternatively, agitating the sample with surface acoustic waves using the ArrayBooster™, a commercially available hybridization instrument from Advalytix, can accelerate hybridization rates as well.

The amplification chip layer has probes and primers for PCR that are appropriate to assay the nucleic acids that the corresponding sample through-holes in the capture chip layer capture. For example, the probes can be designed to capture a particular viral genome or genome fragment and the PCR reagents can amplify one or more sequences within that genome. In a DNA capture and amplification embodiment using wax immobilized reagents, the captured oligo-target nucleic acid pair will melt upon initiation of thermal cycling and the amplification chip layer may have primers that either overlap the capture sequence or are independent. Such an embodiment greatly saves on reagent costs. For example, a standard tube of TaqMan® PCR reagent enables approximately 150,000 tests in such chips.

Use of a prepared layered chip starts with preparation of nucleic acid samples using standard methods of purification and modification. For example, after lysing any potential microbes, the user could use a Qiagen RNA/DNA kit to extract the genomic material, split the sample and perform a random hexamer primed reverse transcription (RT) on a sample fraction, then recombine the two samples. In some embodiments, the RT may be performed on a small fraction of the original sample since viral RNA tends to be present in much higher titers than bacterial DNA.

As in above-described embodiments, the layered chip can be loaded with the prepared sample in a variety of ways. For example, a volume of high-density immersion fluid can be added to a chip holder case that is open on one side. The nucleic acid sample may then be floated in a thin layer on top of the immersion fluid. The prepared chip is then lowered into the chip holder case, and self-loaded with sample as it passes through the sample layer into the immersion fluid. The chip holder case may then be sealed, such as by a sealant that is dispensed on top of the sample and cured.

The capture probes in one of the chip layers, e.g., top chip 31, will interact with and capture the target nucleic acid in the sample liquid. After washing in a buffer to remove non-specifically bound nucleic acids and then replacing the wash buffer with a PCR master-mix (a solution that typically contains polymerase, nucleotides, buffers, magnesium chloride, and dynamic blockers), the layered structure 33 is placed in a thermal cycling system, where elevation of temperature to start a PCR process melts the PEG in the other chip layer, e.g., bottom chip 32, releasing PCR primers and/or probes to commence PCR amplification of the target nucleic acid captured in the through-holes of the other chip.

Imaging/analysis can then be performed on the chip, either in combination with or separately from the thermal cycling processing. Although nucleic acids could alternatively be detected in the chip using end-point PCR, quantitative PCR offers compelling advantages for some applications. After thermal cycling and analysis, the used chip holder case containing the PCR chip and sample can be disposed of.

A complete system to an end-user might include hermetically sealed layered chips that are pre-loaded with capture and PCR primers, along with dilution buffers and master mix, a chip loading and sealing solution, and a compact, inexpensive imaging thermal cycler for real-time PCR. One specific product is based on a 1″×3″ microscope slide-format array chip for use in genotyping by PCR based on end-point analysis. The consumables include a 3072-hole chip and chip case, along with master mix and sealing reagents (perfluorinated liquid and UV curable sealant). With an auto-loading slide scanner and a 20-slide flat block thermal cycler costing less than $100,000, 30,000 SNP analyses per hour can be performed. This is an order of magnitude lower on a SNP per day basis than other systems presently offered, with the added advantage of lower sample consumption.

A layered chip structure can be useful in a variety of other specific applications, for example, detecting a pathogen in a clinical sample. One chip layer can be arranged to capture the target pathogen with an antibody, which may be immobilized on the interior, hydrophilic surface of the chip, and the other chip layer can be arranged for detection of the captured pathogen by PCR amplification. Lysis enzymes such as lysozyme, lipase, or zymolase can be immobilized in wax to aid in lysis of the captured pathogen.

One of the problems with enzyme linked immunosorbant assay (ELISA) arrays is that they currently need to have common assay conditions. A layered chip structure as described above can overcome that, and can also be useful for varying the conditions of ELISA by immobilizing reagents such as buffer salts in wax within one of the chip layers. An ELISA approach may be used in which the pathogen is captured by an antibody immobilized in one part of the through-hole, and a detection antibody is encapsulated in a low-melting point PEG in another part of the through-hole and slowly released into solution. The chip is then rinsed to remove non-bound detection antibodies and the ELISA is developed with secondary antibody conjugated to an enzyme such as alkaline phosphatase or horseradish peroxidase and detected by washing and adding any of the several available chromogenic, flourogenic, or luminescent substrates.

In other examples, capture chip layers can be loaded with DNA hybridization probes for viral RNA and bacterial DNA found in pathogens such as SARS, Influenza A, Influenza B, Respiratory Syncytial Virus, Parainfluenza-1, Parainfluenza-2, Parainfluenza-3 and Bacillus anthracis. Complementary amplification chip layers are then loaded with dry, encapsulated TaqMan® primers and probes to viral nucleic acids sequences expected to be present in the captured viral nucleic acids. The chip layers are bonded and tested for several parameters: detection limits, specificity, quantitative accuracy, chip to chip variability, day to day variability over several months, user to user variability.

While embodiments based on offline sample preparation with oligonucleotide capture and PCR amplification described above are useful in their own right, further embodiments go directly from patient sample to end results with a minimum of operator dependent steps. For example, in one embodiment, multiple viruses can be captured by antibodies in one chip layer, the viruses can be disrupted by temperature and/or enzymatic digestion (while protecting the viral nucleic acids from degradation), and then the lytic enzymes can be denatured (e.g., thermally) and reverse transcription-PCR can be performed. Such an embodiment avoids the need for standard nucleic acid sample-preparation procedures.

Thus, embodiments of the present invention include a reverse transcription system and a PCR amplification system that is encapsulated in multiple chip layers to create an integrated RT-PCR array. Various embodiments also are able to detect low concentrations of multiple pathogen nucleic acid sequences. Specific embodiments also incorporate multiple existing PCR assays for detection of respiratory pathogen nucleic acids including SARS RNA.

Embodiments also provide high test specificity. For example, three probes can be provided for each target DNA sequence; two PCR primers and a capture probe consisting of a complimentary sequence. In some cases, a fourth probes such as a Taqman® probe or molecular beacon may also be used. This reduces the occurrence of false positives and false negatives. Thus, the ability to perform PCR in a high density microfluidic array format can provide superior data quality as compared to conventional DNA microarrays. Additionally, multiple sequences per pathogen can be easily assayed to further increase reliability and decrease the consequences of pathogen mutation.

In addition, specific embodiments have the ability to detect multiple pathogens. By performing reactions in parallel, one-pot multiplex reagents do not have to be developed. Conventional multiplexing either makes use of multiple dyes, which usually allows the detection of just two or three sequences, or a post-processing step such as electrophoresis which adds cost and complexity.

Furthermore, embodiments are well-suited for point-of-care use. The low cost, compact size, and ease of use of specific embodiments enables multiplexed PCR-based assays to be performed in many clinical and point-of-care settings. The greatly reduced primer and probe volumes and the low cost materials and processing methods that have been developed enable a low cost solution for widespread use.

Embodiments are also very scalable, to permit performing a smaller or larger number of measurements per patient sample and/or to process multiple patient samples in parallel. Specific embodiments support chip formats containing up to 24,576 probes or samples. Multiple layered chips can be processed in parallel in a manner analogous to conventional DNA microarrays. Advanced concepts for capture/hybridization may simplify upstream purification processes and enable future integrated devices.

Once produced, layered structure chips typically will be packaged and stored for a reasonable amount of time-perhaps several months-depending on the overall chip format such as the presence of encapsulated proteins and antibodies. Formulations with various stabilizers such as sugars and anti-oxidants may be beneficial. Vacuum packaging and packaging in inert gas with various moisture contents could also be useful, as could cold or frozen storage.

Calibration Dye Drydown

When performing real time PCR, it is common to include a calibration dye, for example, ROX in a TaqMan reaction. The calibration dye corrects for uniformity defects in the excitation and emission optics of the system. In TaqMan reactions, signals are often expressed as a ratio of VIC to ROC fluorescent intensity. When using the through-hole arrays for PCR, it is desirable to correct not just for optical defects but for non-uniformity associate with the loading, drying and re-solubalization of the PCR probes and/or primers on the microfluidic array. This may be accomplished by adding the calibration dye to the primer/Polyethylene Glycol (PEG) mixture prior to drying down. In practice, the calibration dye signal tends to approach a constant value after several initial thermal cycles. In various embodiments, a normalization value measured after several cycles after reaching equilibirium for improved measurements.

In various embodiments, the disclosed system and method may be implemented as a computer program product for use with a computer system. Such implementation may include a series of computer instructions fixed either on a tangible medium, such as a computer readable media (e.g., a diskette, CD-ROM, ROM, or fixed disk) or transmittable to a computer system, via a modem or other interface device, such as a communications adapter connected to a network over a medium. Medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable media with accompanying printed or electronic documentation (e.g., shrink wrapped software), preloaded with a computer system (e.g., on system ROM or fixed disk), or distributed from a server or electronic bulletin board over the network (e.g., the Internet or World Wide Web).

Although various exemplary embodiments of the invention are disclosed below, it should be apparent to those skilled in the art that various changes and modifications can be made that will achieve some of the advantages of the invention without departing from the true scope of the invention.

What is claimed is:

1. A system for thermal cycling a plurality of samples, the system comprising:
a case having a fluid-tight cavity defining an interior volume;
a microfluidic array disposed in the interior volume, the array including a sheet of material having a pair of opposed surfaces, a thickness, and a plurality of through-holes running through the thickness between the surfaces; and
a thermal cycler having at least one thermally controlled surface adapted to thermally contact the case.

2. The system according to claim 1, further comprising a positioning mechanism for retaining the case in a specified position and orientation when thermally contacting the thermally controlled surface.

3. The system according to claim 2, wherein the positioning mechanism includes on the thermally controlled surface one of a protrusion and an indention.

4. The system according to claim 3, wherein the indentation includes a graded surface, such that the microfluidic sample array can be slid into the indentation.

5. The system according to claim 1, wherein the thermal cycler includes a deck for placing the case prior to loading and/or removal from the thermally controlled surface.

6. The system according to claim 5, wherein the case is capable of being slid from the deck onto the thermally controlled surface.

7. The system according to claim 5, wherein the deck is capable of being rotated along a plane of the thermally controlled surface.

8. The system according to claim 1, wherein the thermal cycler includes a finger element for pressing the case against the thermally controlled surface.

9. The system according to claim 1, further comprising a heat transfer pad positioned between the case and the thermally controlled surface.

10. The system according to claim 1, further comprising an illumination source capable of illuminating at least one of the through-holes at one or more defined wavelengths.

11. The system according to claim 10, wherein the illumination source includes at least one LED.

12. The system according to claim 10, further comprising an imaging device for imaging one or more through-holes to provide imaging data, and wherein the illumination source includes at least two illuminations sources symmetrically positioned off-axis from the camera with reference to the array.

13. The system according to claim 1, further comprising an imaging device for imaging one or more through-holes to provide imaging data.

14. The system according to claim 13, wherein the imaging device is one of a camera and a a scanner, the camera for simultaneously imaging each of the through-holes to provide imaging data, the scanner for imaging one or more of the through-holes sequentially to provide imaging data.

15. The system according to claim 1, further comprising:
an immersion fluid disposed in the interior volume.

16. The system according to claim 1, wherein the array has greater than 100 through-holes.

17. The system according to claim 1, wherein the array has a through-hole density greater than one through-hole per 20 mm².

18. The system according to claim 1, further comprising:
an enclosure, the thermal cycler positioned within the enclosure;
an imaging device positioned within the enclosure for imaging the sample; and
an illumination system positioned with the enclosure for illuminated at least one sample at one or more predefined wavelengths.

19. A method of thermal cycling a plurality of samples, the method comprising:
holding a microfluidic array in a fluid-tight cavity in a case, the array including a sheet of material having a pair of opposed surfaces, a thickness, and a plurality of through-holes running through the thickness between the surfaces; and
placing the case in thermal contact with a thermally controlled surface.

20. The method according to claim 19, further comprising covering the microfluidic array in the cavity with a volume of an immersion fluid.

21. The method according to claim 19, further comprising using a positioning mechanism for retaining the case in a specified position and orientation when thermally contacting the thermally controlled surface.

22. The method according to claim 19, further comprising illuminating the at least one of the through-holes at one or more defined wavelengths.

23. The method according to claim 22, further comprising imaging at least one through-hole.

24. The method according to claim 23, wherein illuminating includes providing illumination from at two illumination sources symmetrically positioned off-axis from the imaging device with reference to the array.

25. The method according to claim 23, wherein imaging includes sequentially imaging two or more through-holes.

26. The method according to claim 23, wherein imaging includes imaging each through-hole simultaneously.

27. The method according to claim 19, wherein the array has greater than 100 through-holes.

28. The method according to claim 19, wherein the array has a through-hole density greater than one through-hole per 20 mm².
